(19) United States
(12) Patent Application Publication
(10) Pub. No.: US 2023/0160019 A1
(43) Pub. Date: May 25, 2023

(54) RNA MARKERS AND METHODS FOR IDENTIFYING COLON CELL PROLIFERATIVE DISORDERS

(71) Applicant: Freenome Holdings, Inc., South San Francisco, CA (US)

(72) Inventors: Frances ARMSTRONG, San Francisco, CA (US); Shivani MAHAJAN, San Francisco, CA (US); Adam HARVEY, San Mateo, CA (US); Aneesha TEWARI, San Francisco, CA (US); David WEINBERG, San Francisco, CA (US); Jesse EATON, San Francisco, CA (US)

(21) Appl. No.: 18/049,785

(22) Filed: Oct. 26, 2022

Related U.S. Application Data
(63) Continuation of application No. PCT/US2021/029361, filed on Apr. 27, 2021.
(60) Provisional application No. 63/024,875, filed on May 14, 2020.
(60) Provisional application No. 63/017,552, filed on Apr. 29, 2020.

(51) Int. Cl. C12Q 1/6886; G16B 40/20

(52) U.S. Cl. CPC C12Q 1/6886; G16B 40/20; C12Q 2600/178; C12Q 2600/158

(57) ABSTRACT

The present disclosure pertains to miRNAs that are differentially expressed in samples of an individual having colon cell proliferative disorder, or having a high risk of developing colon cell proliferative disorder, as compared to the corresponding sample of an individual not having colon cell proliferative disorder, or having low risk of developing colon cell proliferative disorder, respectively. In some embodiments, the miRNAs are differentially expressed in a tissue sample or blood plasma sample of an individual having a colorectal lesion and having a high risk of developing colon cell proliferative disorder as compared to the corresponding tissue sample or blood sample of an individual having the colorectal lesion and having no risk or low risk of developing colon cell proliferative disorder. These differentially expressed miRNAs can be used as biomarkers for diagnosis, treatment, and/or prevention of a colon cell proliferative disorder, particularly, in a subject having a colorectal lesion.

### CROSS-REFERENCE TO RELATED APPLICATIONS

This application is a continuation of International Patent Application No. PCT/US2021/029361, filed Apr. 27, 2021, which claims the benefit of U.S. Provisional Patent Application No. 63/017,552, filed Apr. 29, 2020, and U.S. Provisional Patent Application No. 63/024,875, filed May 14, 2020, each of which is incorporated by reference herein in its entirety.

### BACKGROUND

Cancer screening and early detection are considered the most efficient strategies against cancer because detecting malignancy or a precursor lesion at an early stage prior to the onset of symptoms are when treatments are most effective. In colorectal cancer, for instance, colonoscopies play a role in improving early diagnosis. While colonoscopies are useful for early detection, patient compliance rates are low, and screening is conducted below recommended regularity due to the invasiveness of the procedure. Thus, non-invasive methods offer a more promising approach for early cancer detection.

### SUMMARY

The present disclosure provides methods and systems directed to micro ribonucleic acid (microRNA, or miRNA) profiling of genes associated with colon cell proliferative disorder (e.g., colorectal cancer) detection and disease progression. Some embodiments of the present disclosure provide miRNAs that are differentially abundant in a sample of a subject having a colon cell proliferative disorder, or having a high risk of developing colon cell proliferative disorder, as compared to the corresponding sample of a subject not having colon cell proliferative disorder, or having low risk of developing colon cell proliferative disorder. In some embodiments, each of the subjects having high risk of developing colon cell proliferative disorder and the subjects having low risk of developing colon cell proliferative disorder have a non-invasive precursor lesion arising within colorectal mucosa (hereinafter, colorectal lesion). The miRNAs that are present at different abundances (often referred to as “differentially expressed”) in a sample of a subject having colon cell proliferative disorder, or having a high risk of developing colon cell proliferative disorder, can be used as biomarkers for diagnosis, treatment, and/or prevention of colon cell proliferative disorder.

The miRNAs identified herein can be used to identify subjects that have colon cell proliferative disorder to distinguish them from subjects that do not have colon cell proliferative disorder, or to identify subjects having a higher risk of developing colon cell proliferative disorder to distinguish them from subjects that have a lower risk of developing colon cell proliferative disorder, or to identify subjects having a colon cell proliferative disorder precursor (such as intraductal papillary mucinous neoplasm (IPMN)) versus a non-IPMN, or to identify subjects that have a malignant IPMN versus a benign IPMN. Thus, these miRNAs can be used as an adjunctive tool to guide decisions regarding monitoring, treatment, and management of colon cell proliferative disorder.

Some embodiments of the present disclosure provide a machine learning model classifier trained on the miRNAs described herein that are differentially expressed in a sample of a subject having colon cell proliferative disorder, or having a high risk of developing colon cell proliferative disorder, for example, when the subject has a colorectal lesion. In an example, a method is provided for a blood-based minimally-invasive miRNA assay that can be used in a subject having a colorectal lesion to assess histologic severity. In another embodiment, the miRNAs indicative of colon cell proliferative disorder are detected in cell-free samples from a subject, for example, bodily fluid samples from a subject, such as whole blood, plasma, or serum. As such, the present disclosure provides miRNAs that can be used to differentiate between the presence or absence of colon cell proliferative disorder, high-risk or low-risk colorectal lesions that warrant treatment such as, surgical resection, immunotherapy, radiation, or chemotherapy) and low-risk colorectal lesions that can be monitored. Monitoring and confirmation of the presence of colon cell proliferative disorder or lesions can be carried out, for example, by colonoscopy, ultrasound, MM, or CT scan.

In an aspect, the present disclosure provides a micro ribonucleic acid (miRNA) signature panel characteristic of a colon cell proliferative disorder, comprising: a pre-determined set of one or more, two or more, three or more, or four or more miRNAs selected from the group listed in Tables 1-11, wherein the four or more miRNAs are differentially expressed between a biological sample from a subject having the colon cell proliferative disorder or subtype thereof, and a biological sample from a subject without the colon cell proliferative disorder or subtype thereof

In some embodiments, the miRNA signature panel is characteristic of advanced adenoma, and the signature panel comprises: a pre-determined set of miRNAs comprising: a) hsa-miR-1273a, hsa-miR-17-5p, hsa-miR-20a-3p, hsa-miR-20b-5p; b) hsa-miR-3065-5p, hsa-miR-4785, hsa-miR-5096, hsa-miR-5189-5p, or c) hsa-miR-545-3p, hsa-miR-570-3p, hsa-miR-624-3p, hsa-mir-1181, hsa-mir-6073, wherein the miRNAs are differentially expressed between a biological sample from a subject having advanced adenoma or subtype thereof, and a biological sample from a subject without advanced adenoma or subtype thereof.

In some embodiments, the miRNA signature panel is characteristic of colorectal cancer, and the signature panel comprises: a pre-determined set of miRNAs comprising: a) hsa-miR-1250-5p, hsa-miR-1255a, hsa-miR-223-3p, hsa-miR-338-3p, hsa-miR-338-5p; b) hsa-miR-424-5p, hsa-miR-424-3p, hsa-miR-450a-5p, hsa-miR-450b-5p, hsa-miR-4772-3p; c) hsa-miR-4772-5p, hsa-miR-625-5p, hsa-miR-7847-3p, hsa-miR-1181, hsa-miR-3651, hsa-mir-6073; d) hsa-mir-6125, hsa-mir-7704, hsa-miR-19b-3p, hsa-miR-19a-3p, hsa-miR-3157-5p; e) hsa-miR-142-3p, hsa-miR-30c-5p, hsa-miR-6741-5p, hsa-miR-590-3p, hsa-miR-4685-5p; hsa-miR-3648, hsa-miR-331-3p, hsa-miR-1303, hsa-miR-6790-3p, hsa-miR-6867-5p, hsa-miR-942-5p; g) hsa-miR-378a-3p, hsa-miR-1287-5p, hsa-mir-4785, hsa-miR-324-3p, hsa-miR-550b-2-5p; h) hsa-miR-200c-3p, hsa-miR-200b-3p, hsa-miR-3679-5p, hsa-miR-550a-3-5p, hsa-miR-3187-3p; i) hsa-miR-181b-5p, hsa-miR-3138, hsa-miR-146a-5p, hsa-miR-6721-5p, hsa-miR-23b-3p, hsa-miR-28-5p; j) hsa-miR-320d, hsa-miR-940, hsa-miR-320d-1, hsa-miR-10a-5p, hsa-miR-340-5p; k) hsa-miR-320b, hsa-miR-335-5p, hsa-miR-320c, hsa-miR-501-3p, hsa-miR-548n; or l) hsa-miR-27a-3p, hsa-miR-3065-3p, hsa-miR-548aa@, hsa-miR-584-3p, hsa-miR-22-3p, wherein the miRNAs are differentially expressed between a biological sample from a subject having the colorectal cancer or subtype thereof, and a biological sample from a subject without the colorectal cancer or subtype thereof.

In some embodiments, the pre-determined set of miRNAs comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 250 miRNAs selected from the group listed in Tables 1-11.

In some embodiments, the biological sample is selected from the group consisting of bodily fluid, stool, colonic effluent, urine, blood plasma, blood serum, whole blood, isolated blood cells, cells isolated from the blood, and combinations thereof.

In some embodiments, the biological sample comprises a nucleic acid, DNA, RNA, or cell-free nucleic acid (cfDNA or cfRNA).

In some embodiments, the miRNA comprises mature miRNAs and miRNA hairpins.

In some embodiments, the signature panel comprises differential expression in 1 or more, 2 or more, 3 or more, 4 or more, 5 or more, 6 or more, 7 or more, 8 or more, 9 or more, 10 or more, 11 or more, or 12 or more miRNAs selected from the group listed in Tables 1-11.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the colon cell proliferative disorder is selected from the group consisting of stage 1 colorectal cancer, stage 2 colorectal cancer, stage 3 colorectal cancer, and stage 4 colorectal cancer.

In another aspect, the present disclosure provides a classifier capable of distinguishing a population of healthy subjects (e.g., subjects without colon cell proliferative disorder) from subjects with colon cell proliferative disorder, comprising: a) sets of measured values representative of differential miRNA expression in 6 or more, or 12 or more pre-selected miRNAs selected from the group listed in Tables 1-11, wherein the measured values are obtained from miRNA expression data from healthy subjects and subjects having a colon cell proliferative disorder, b) wherein the measured values are used to generate a set of features corresponding to properties of the differential miRNA expression, and wherein the set of features is computer processed using machine learning model (e.g., a statistical model), c) wherein the machine model provides a feature vector useful as a classifier capable of distinguishing a population of healthy subjects from subjects having a colon cell proliferative disorder.

In some embodiments, the pre-selected miRNAs comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 250 miRNAs selected from the group listed in Tables 1-11.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the sets of measured values describe characteristics of the differential miRNA expression selected from the group consisting of: count or rate of observing fragments with different counts, raw miRNA abundance, miRNA abundance normalized to housekeeping genes, miRNA abundance normalized to synthetic sequences, log normalized miRNA abundance, fragment length, fragment midpoint, read mapping position and read pile up along mature miRNAs or miRNA hairpins, and abundances of clusters of miRNAs.

In some embodiments, the machine learning model is trained using training data obtained from training biological samples, a first subset of the training biological samples identified as corresponding to a subject having a colon cell proliferative disorder and a second subset of the training biological samples identified corresponding to a subject as not having a colon cell proliferative disorder.

In some embodiments, the classifier is provided in a system for detecting a colon cell proliferative disorder comprising: a) a computer-readable medium comprising a classifier operable to classify the subjects based on a miRNA signature panel; and b) one or more processors for executing instructions stored on the computer-readable medium.

In some embodiments, the system comprises a classification circuit that is configured as a machine learning classifier selected from the group consisting of a deep learning classifier, a neural network classifier, a linear discriminant analysis (LDA) classifier, a quadratic discriminant analysis (QDA) classifier, a support vector machine (SVM) classifier, a random forest (RF) classifier, a linear kernel support vector machine classifier, a first or second order polynomial kernel support vector machine classifier, a ridge regression classifier, an elastic net algorithm classifier, a sequential minimal optimization algorithm classifier, a naive Bayes algorithm classifier, and principal component analysis classifier.

In another aspect, the present disclosure provides a method for determining a micro ribonucleic acid (miRNA) profile of a biological sample from a subject, comprising: a) isolating RNA molecules from the biological sample; b) ligating RNA adapters to the RNA molecules, before or after reverse transcribing the RNA molecules to complementary deoxyribonucleic acid (cDNA) molecules; c) amplifying the cDNA molecules; d) determining nucleic acid sequences of the cDNA molecules; e) aligning the nucleic acid sequences to a reference nucleic acid sequence for a panel of miRNAs selected from the group listed in Tables 1-11; and f) determining the miRNA profile of the subject based at least in part on the aligned nucleic acid sequences.

In some embodiments, the pre-selected miRNAs comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 250 miRNAs selected from the group listed in Tables 1-11.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the method further comprises enriching or depleting the RNA molecules or the cDNA molecules.

In some embodiments, the reference nucleic acid sequence comprises genome, transcriptome, or custom transcriptome reference nucleic acid sequences.

In some embodiments, the method further comprises preparing a miRNA library before the amplifying.

In some embodiments, the adapter ligation comprises RNA adapter ligation, adapter blocking, adapter circularization and dimer removal before c).

In some embodiments, ligating the RNA adapters comprises performing adapter blocking, adapter circularization, and/or dimer removal.

In some embodiments, ligating the RNA adapters comprises performing 3′ RNA adapter ligation, 5′ RNA adapter ligation, reverse transcription with unique molecular identifier (UMI) assignment, and/or cDNA cleanup.

In another aspect, the present disclosure provides a method for determining a micro ribonucleic acid (miRNA) profile of a biological sample from a subject, comprising performing one or more of: 1) Extraction of RNA molecules from the biological sample followed by direct RNA counting, 2) Extraction of RNA molecules from the biological sample followed by A tailing, then reverse transcribing (RT) to cDNA with template switching, 3) Extraction of RNA molecules from the biological sample followed by A tailing, then reverse transcription polymerase chain reaction (RT-PCR) and quantitative PCR (qPCR) or digital droplet PCR (ddPCR), 4) Extraction of RNA molecules from the biological sample followed by sequence-specific ligation, and then RT-PCR and qPCR or ddPCR, and 5) Extraction-free miRNA profiling of RNA molecules from the biological sample in absence of performing RNA isolation; and determining the miRNA profile of the biological sample from the subject.

In some embodiments, determining the miRNA profile comprises use of a reference nucleic acid sequence that is part of a human genome or human transcriptome database.

In some embodiments, determining the miRNA profile comprises generating a counts table of expressed miRNA.

In some embodiments, determining the miRNA profile comprises generating a counts table normalized based on expressed miRNA to identify differentially-abundant miRNA.

In some embodiments, the miRNA profile is associated with a colon cell proliferative disorder and provides classification of a subject as having a colon cell proliferative disorder or not having a colon cell proliferative disorder.

In some embodiments, the biological sample from the subject is selected from the group consisting of bodily fluid, stool, colonic effluent, urine, blood plasma, blood serum, whole blood, isolated blood cells, cells isolated from the blood, and combinations thereof.

In some embodiments, the method further comprises comparing the miRNA profile against a database of reference miRNA profiles from healthy subjects; and determining that the subject has an increased risk of having a colon cell proliferative disorder based at least in part on measuring a change of at least 5%, at least 10%, at least 15%, at least 20%, at least 25%, at least 30%, at least 35%, at least 40%, at least 45%, or at least 50% in miRNA expression of the miRNA profile relative to the reference miRNA profiles.

In some embodiments, the method further comprises comparing the miRNA profile against a database of reference miRNA profiles from healthy subjects; and determining that the subject has an increased risk of having a colon cell proliferative disorder based at least in part on measuring a change of at least 15% in miRNA expression of the miRNA profile relative to the reference miRNA profiles.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the colon cell proliferative disorder is selected from the group consisting of stage 1 colorectal cancer, stage 2 colorectal cancer, stage 3 colorectal cancer, and stage 4 colorectal cancer.

In some embodiments, the advanced adenoma comprises a tubular adenoma, a tubulovillous adenoma, a villous adenoma, an adenocarcinoma, or a hyperplastic polyp.

In another aspect, the present disclosure provides a method for detecting a presence or an absence of a colon cell proliferative disorder in a subject, comprising: a) isolating ribonucleic acid (RNA) molecules from the biological sample; b) ligating RNA adapters to the RNA molecules, before or after reverse transcribing the RNA molecules to complementary deoxyribonucleic acid (cDNA) molecules; c) amplifying the cDNA molecules; d) determining nucleic acid sequences of the cDNA molecules; e) aligning the nucleic acid sequences to a reference nucleic acid sequence for a pre-identified panel of miRNAs selected from the group listed in Tables 1-11; f) determining an miRNA profile based at least in part on the aligned nucleic acid sequences; and g) computer processing the miRNA profile using a machine learning model trained to be capable of distinguishing between healthy subjects and subjects with a colon cell proliferative disorder to provide an output value associated with presence or absence of a colon cell proliferative disorder, thereby indicating the presence or the absence of the colon cell proliferative disorder in the subject.

In some embodiments, b) comprises incorporating sample-specific barcodes and/or molecule-specific unique molecular identifiers (UMIs).

In some embodiments, the pre-selected miRNAs comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 250 miRNAs selected from the group listed in Tables 1-11.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the reference nucleic acid sequence is part of a human genome or human transcriptome database.

In some embodiments, determining the miRNA profile of the subject comprises generating a counts table of expressed miRNA.

In some embodiments, determining the miRNA profile of the subject comprises generating a counts table of expressed miRNA to identify differentially-abundant miRNA.

In some embodiments, the biological sample from the subject is selected from the group consisting of bodily fluid, stool, colonic effluent, urine, blood plasma, blood serum, whole blood, isolated blood cells, cells isolated from the blood, and combinations thereof.

In some embodiments, the method further comprises comparing the miRNA profile against a database of reference miRNA profiles from healthy subjects; and determining that the subject has an increased risk of having a colon cell proliferative disorder based at least in part on measuring a change of at least 5%, at least 10%, at least 15%, at least 20%, at least 25%, at least 30%, at least 35%, at least 40%, at least 45%, or at least 50% in the miRNA expression of the miRNA profile relative to the reference miRNA profiles.

In some embodiments, the method further comprises comparing the miRNA profile against a database of reference miRNA profiles from healthy subjects; and determining that the subject has an increased risk of having a colon cell proliferative disorder based at least in part on measuring a change of at least 15% in the miRNA expression of the miRNA profile relative to the reference miRNA profiles.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the colon cell proliferative disorder is selected from the group consisting of stage 1 colorectal cancer, stage 2 colorectal cancer, stage 3 colorectal cancer, and stage 4 colorectal cancer.

In another aspect, the present disclosure provides a method for determining a miRNA profile of a biological sample from a subject comprising: a) isolating ribonucleic acid (RNA) molecules from the biological sample; b) reverse transcribing the RNA molecules to complementary deoxyribonucleic acid (cDNA) molecules; c) ligating RNA adapters to the RNA molecules or the cDNA molecules; d) amplifying the cDNA molecules; e) determining nucleic acid sequences of the cDNA molecules; f) aligning the nucleic acid sequences to a reference nucleic acid sequence for a panel of miRNAs selected from the group listed in Tables 1-11; and g) determining the miRNA profile based at least in part on the aligned nucleic acid sequences.

In some embodiments, the pre-selected miRNAs comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 250 miRNAs selected from the group listed in Tables 1-11.

In some embodiments, the method further comprises administering a treatment to the subject based on the detected colon cell proliferative disorder. In some embodiments, the treatment comprises chemotherapy, radiotherapy, immunotherapy, or surgery.

In another aspect, the present disclosure provides a method for detecting a presence or an absence of a colon cell proliferative disorder in a subject, comprising: a) isolating ribonucleic acid (RNA) molecules from the biological sample; b) reverse transcribing the RNA molecules to complementary deoxyribonucleic acid (cDNA) molecules; c) ligating RNA adapters to the RNA molecules or the cDNA molecules; d) amplifying the cDNA molecules; e) determining nucleic acid sequences of the cDNA molecules; f) aligning the nucleic acid sequences to a reference nucleic acid sequence for a panel of miRNAs selected from the group listed in Tables 1-11; g) determining an miRNA profile based at least in part on the aligned nucleic acid sequences; h) computer processing the miRNA profile using a machine learning model trained to distinguish between subjects not having the colon cell proliferative disorder and subjects having the colon cell proliferative disorder; and i) outputting by the machine learning model a value associated with subjects having the colon cell proliferative disorder or with subjects not having the colon cell proliferative disorder, thereby detecting the presence or the absence of the colon cell proliferative disorder in the subject.

In some embodiments, the pre-selected miRNAs comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 250 miRNAs selected from the group listed in Tables 1-11.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the method further comprises administering a treatment to the subject based on the detected colon cell proliferative disorder. In some embodiments, the treatment comprises chemotherapy, radiotherapy, immunotherapy, or surgery.

In another aspect, the present disclosure provides a method for monitoring minimal residual disease in a subject previously treated for a disease, comprising: determining a micro ribonucleic acid (miRNA) profile of a biological sample from the subject using a panel of miRNAs selected from the group listed in Tables 1-11, thereby generating a baseline miRNA state; determining a miRNA profile of a biological sample obtained from the subject at one or more time points after the generating of the baseline miRNA state, thereby generating a current miRNA state; and determining a difference between the baseline miRNA state and the current miRNA state, thereby detecting a change in the minimal residual disease in the subject.

In some embodiments, the minimal residual disease is selected from the group consisting of response to treatment, tumor load, residual tumor post-surgery, relapse, secondary screen, primary screen, and cancer progression. In some embodiments, the method further comprises administering a treatment to the subject based on the detected change in the minimal residual disease in the subject. In some embodiments, the treatment comprises chemotherapy, radiotherapy, immunotherapy, or surgery.

In another aspect, a method is provided for determining response of a subject to treatment, comprising: determining a micro ribonucleic acid (miRNA) profile of a biological sample from the subject using a panel of miRNAs selected from the group listed in Tables 1-11, thereby generating a baseline miRNA state; determining a miRNA profile of a biological sample obtained from the subject at one or more time points after the generating of the baseline miRNA state, thereby generating a current miRNA state; and determining a difference between the baseline miRNA state and the current miRNA state, thereby determining the response of the subject to the treatment. In some embodiments, the method further comprises administering a treatment to the subject based on the determined response of the subject to the treatment. In some embodiments, the treatment comprises chemotherapy, radiotherapy, immunotherapy, or surgery.

In another aspect, a method is provided for monitoring tumor load of a subject, comprising: determining a micro ribonucleic acid (miRNA) profile of a biological sample from the subject using a panel of miRNAs selected from the group listed in Tables 1-11, thereby generating a baseline miRNA state; determining a miRNA profile of a biological sample obtained from the subject at one or more time points after the generating of the baseline miRNA state, thereby generating a current miRNA state; and determining a difference between the baseline miRNA state and the current miRNA state, thereby monitoring the tumor load of the subject. In some embodiments, the method further comprises administering a treatment to the subject based on the tumor load of the subject. In some embodiments, the treatment comprises chemotherapy, radiotherapy, immunotherapy, or surgery.

In another aspect, a method is provided for detecting residual tumor post-surgery of a subject, comprising: determining a micro ribonucleic acid (miRNA) profile of a biological sample from the subject using a panel of miRNAs selected from the group listed in Tables 1-11, thereby generating a baseline miRNA state; determining a miRNA profile of a biological sample obtained from the subject at one or more time points after the generating of the baseline miRNA state, thereby generating a current miRNA state; and determining a difference between the baseline miRNA state and the current miRNA state, thereby detecting the residual tumor post-surgery of the subject. In some embodiments, the method further comprises administering a treatment to the subject based on the residual tumor post-surgery of the subject. In some embodiments, the treatment comprises chemotherapy, radiotherapy, immunotherapy, or surgery.

In another aspect, a method is provided for detecting relapse of a subject, comprising: determining a micro ribonucleic acid (miRNA) profile of a biological sample from the subject using a panel of miRNAs selected from the group listed in Tables 1-11, thereby generating a baseline miRNA state; determining a miRNA profile of a biological sample obtained from the subject at one or more time points after the generating of the baseline miRNA state, thereby generating a current miRNA state; and determining a difference between the baseline miRNA state and the current miRNA state, thereby detecting the relapse of the subject. In some embodiments, the method further comprises administering a treatment to the subject based on the detected relapse of the subject. In some embodiments, the treatment comprises chemotherapy, radiotherapy, immunotherapy, or surgery.

In another aspect, a method is provided for performing a secondary screen, based at least in part on a miRNA profile of a subject.

In another aspect, a method is provided for performing a primary screen, based at least in part on a miRNA profile of a subject.

In another aspect, a method is provided for monitoring cancer progression of a subject, comprising: determining a micro ribonucleic acid (miRNA) profile of a biological sample from the subject using a panel of miRNAs selected from the group listed in Tables 1-11, thereby generating a baseline miRNA state; determining a miRNA profile of a biological sample obtained from the subject at one or more time points after the generating of the baseline miRNA state, thereby generating a current miRNA state; and determining a difference between the baseline miRNA state and the current miRNA state, thereby monitoring the cancer progression of the subject. In some embodiments, the method further comprises administering a treatment to the subject based on the cancer progression of the subject. In some embodiments, the treatment comprises chemotherapy, radiotherapy, immunotherapy, or surgery.

In some embodiments, the miRNA profile is indicative of a presence or susceptibility of colorectal cancer in the subject at a sensitivity of at least about 40%. In some embodiments, the miRNA profile is indicative of a presence or susceptibility of colorectal cancer in the subject at a sensitivity of at least about 50%. In some embodiments, the miRNA profile is indicative of a presence or susceptibility of colorectal cancer in the subject at a sensitivity of at least about 60%. In some embodiments, the miRNA profile is indicative of a presence or susceptibility of colorectal cancer in the subject at a sensitivity of at least about 70%. In some embodiments, the miRNA profile is indicative of a presence or susceptibility of colorectal cancer in the subject at a sensitivity of at least about 80%. In some embodiments, the miRNA profile is indicative of a presence or susceptibility of colorectal cancer in the subject at a sensitivity of at least about 90%. In some embodiments, the miRNA profile is indicative of a presence or susceptibility of colorectal cancer in the subject at a sensitivity of at least about 95%.

In some embodiments, the miRNA profile is indicative of a presence or susceptibility of colorectal cancer in the subject at a positive predictive value (PPV) of at least about 30%. In some embodiments, the miRNA profile is indicative of a presence or susceptibility of colorectal cancer at a positive predictive value (PPV) of at least about 40%. In some embodiments, the miRNA profile is indicative of a presence or susceptibility of colorectal cancer at a positive predictive value (PPV) of at least about 50%. In some embodiments, the miRNA profile is indicative of a presence or susceptibility of colorectal cancer at a positive predictive value (PPV) of at least about 60%. In some embodiments, the miRNA profile is indicative of a presence or susceptibility of colorectal cancer at a positive predictive value (PPV) of at least about 70%. In some embodiments, the miRNA profile is indicative of a presence or susceptibility of colorectal cancer at a positive predictive value (PPV) of at least about 80%. In some embodiments, the miRNA profile is indicative of a presence or susceptibility of colorectal cancer at a positive predictive value (PPV) of at least about 90%. In some embodiments, the miRNA profile is indicative of a presence or susceptibility of colorectal cancer at a positive predictive value (PPV) of at least about 95%. In some embodiments, the miRNA profile is indicative of a presence or susceptibility of colorectal cancer at a positive predictive value (PPV) of at least about 99%.

In some embodiments, the miRNA profile is indicative of a presence or susceptibility of colorectal cancer at a negative predictive value (NPV) of at least about 40%. In some embodiments, the miRNA profile is indicative of a presence or susceptibility of colorectal cancer at a negative predictive value (NPV) of at least about 50%. In some embodiments, the miRNA profile is indicative of a presence or susceptibility of colorectal cancer at a negative predictive value (NPV) of at least about 60%. In some embodiments, the miRNA profile is indicative of a presence or susceptibility of colorectal cancer at a negative predictive value (NPV) of at least about 70%. In some embodiments, the miRNA profile is indicative of a presence or susceptibility of colorectal cancer at a negative predictive value (NPV) of at least about 80%. In some embodiments, the miRNA profile is indicative of a presence or susceptibility of colorectal cancer at a negative predictive value (NPV) of at least about 90%. In some embodiments, the miRNA profile is indicative of a presence or susceptibility of colorectal cancer at a negative predictive value (NPV) of at least about 95%. In some embodiments, the miRNA profile is indicative of a presence or susceptibility of colorectal cancer at a negative predictive value (NPV) of at least about 99%.

In some embodiments, the trained algorithm determines a presence or susceptibility of colorectal cancer of the subject with an Area Under Curve (AUC) of at least about 0.50. In some embodiments, the trained algorithm determines a presence or susceptibility of colorectal cancer of the subject with an Area Under Curve (AUC) of at least about 0.60. In some embodiments, the trained algorithm determines a presence or susceptibility of colorectal cancer of the subject with an Area Under Curve (AUC) of at least about 0.70. In some embodiments, the trained algorithm determines a presence or susceptibility of colorectal cancer of the subject with an Area Under Curve (AUC) of at least about 0.80. In some embodiments, the trained algorithm determines a presence or susceptibility of colorectal cancer of the subject with an Area Under Curve (AUC) of at least about 0.90. In some embodiments, the trained algorithm determines a presence or susceptibility of colorectal cancer of the subject with an Area Under Curve (AUC) of at least about 0.95. In some embodiments, the trained algorithm determines a presence or susceptibility of colorectal cancer of the subject with an Area Under Curve (AUC) of at least about 0.99.

In some embodiments, the method further comprises presenting a report or a graphical user interface of an electronic device of a user. In some embodiments, the user is the subject, individual, or patient.

In some embodiments, the method further comprises determining a likelihood of the determination of a presence or susceptibility of colorectal cancer in the subject, individual, or patient.

In some embodiments, the trained algorithm (e.g., machine learning model or classifier) comprises a supervised machine learning algorithm. In some embodiments, the supervised machine learning algorithm comprises a deep learning algorithm, a support vector machine (SVM), a neural network, or a Random Forest.

In some embodiments, the method further comprises providing the subject with a therapeutic intervention based at least in part on the miRNA profile or analysis, such as a therapeutic intervention to treat a patient with colorectal cancer (e.g., chemotherapy, radiotherapy, immunotherapy, or surgery).

In some embodiments, the method further comprises monitoring the presence or susceptibility of the colorectal cancer, wherein the monitoring comprises assessing the presence or susceptibility of the colorectal cancer of said subject at a plurality of time points, wherein the assessing is based at least on the presence or susceptibility of the colorectal cancer determined at each of the plurality of time points.

In some embodiments, a difference in the assessment of the presence or susceptibility of the colorectal cancer of the subject among the plurality of time points is indicative of one or more clinical indications selected from the group consisting of: (i) a diagnosis of the presence or susceptibility of the colorectal cancer of the subject, (ii) a prognosis of the presence or susceptibility of the colorectal cancer of the subject, and (iii) an efficacy or non-efficacy of a course of treatment for treating the presence or susceptibility of the colorectal cancer of the subject.

In some embodiments, the method further comprises stratifying the colorectal cancer of the subject by using the trained algorithm to determine a sub-type of the colorectal cancer of the subject from among a plurality of distinct subtypes or stages of colorectal cancer.

Another aspect of the present disclosure provides a classifier for distinguishing a population of subjects having a colon cell proliferative disorder from subjects not having the colon cell proliferative disorder comprising: sets of measured values representative of differential miRNA abundance in 6 or more miRNAs selected from the group listed in Tables 1-11, wherein the measured values are obtained from miRNA expression data from subjects not having the colon cell proliferative disorder and subjects having the colon cell proliferative disorder, wherein the measured values are used to generate a set of features corresponding to properties of the differential miRNA abundance and wherein the features are incorporated into a machine learning or statistical model, wherein the machine learning or statistical model provides a feature vector useful as a classifier capable of distinguishing a population of subjects not having the colon cell proliferative disorder from subjects having a colon cell proliferative disorder.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Another aspect of the present disclosure provides a system comprising: a) a computer-readable medium comprising a classifier for distinguishing a population of subjects having a colon cell proliferative disorder from subjects not having the colon cell proliferative disorder based on a miRNA signature panel using a machine learning model; and b) one or more processors for executing instructions stored on the computer-readable medium.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Examples of the present disclosure will now be described, by way of example only, with reference to the attached Figures. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 provides a schematic of a computer system that is programmed or otherwise configured with the machine learning models and classifiers in order to implement methods provided herein.

FIG. 2 provides a histogram showing miRNAs selected during a feature selection.

FIG. 3 provides a graph showing logistic regression coefficients of the top 10 most frequently selected miRNAs.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Cancer screening and early detection are considered the most efficient strategies against cancer because detecting malignancy or a precursor lesion at an early stage prior to the onset of symptoms are when treatments are most effective. In colorectal cancer, for instance, colonoscopies play a role in improving early diagnosis. While colonoscopies are useful for early detection, patient compliance rates are low, and screening is conducted below recommended regularity due to the invasiveness of the procedure. Thus, non-invasive methods offer a more promising approach for early cancer detection.

The present disclosure provides methods and systems directed to micro ribonucleic acid (microRNA, or miRNA) profiling of genes associated with colon cell proliferative disorder (e.g., colorectal cancer) detection and disease progression. Some embodiments of the present disclosure provide miRNAs that are differentially abundant in a sample of a subject having a colon cell proliferative disorder, or having a high risk of developing colon cell proliferative disorder, as compared to the corresponding sample of a subject not having colon cell proliferative disorder, or having low risk of developing colon cell proliferative disorder. In some embodiments, each of the subjects having high risk of developing colon cell proliferative disorder and the subjects having low risk of developing colon cell proliferative disorder have a non-invasive precursor lesion arising within colorectal mucosa (hereinafter, colorectal lesion). The miRNAs that are present at different abundances (often referred to as “differentially expressed”) in a sample of a subject having colon cell proliferative disorder, or having a high risk of developing colon cell proliferative disorder, can be used as biomarkers for diagnosis, treatment, and/or prevention of colon cell proliferative disorder.

The miRNAs identified herein can be used to identify subjects that have colon cell proliferative disorder to distinguish them from subjects that do not have colon cell proliferative disorder, or to identify subjects having a higher risk of developing colon cell proliferative disorder to distinguish them from subjects that have a lower risk of developing colon cell proliferative disorder, or to identify subjects having a colon cell proliferative disorder precursor (such as intraductal papillary mucinous neoplasm (IPMN)) versus a non-IPMN, or to identify subjects that have a malignant IPMN versus a benign IPMN. Thus, these miRNAs can be used as an adjunctive tool to guide decisions regarding monitoring, treatment, and management of colon cell proliferative disorder.

Some embodiments of the present disclosure provide a machine learning model classifier trained on the miRNAs described herein that are differentially expressed in a sample of a subject having colon cell proliferative disorder, or having a high risk of developing colon cell proliferative disorder, for example, when the subject has a colorectal lesion. In an example, a method is provided for a blood-based minimally-invasive miRNA assay that can be used in a subject having a colorectal lesion to assess histologic severity. In another embodiment, the miRNAs indicative of colon cell proliferative disorder are detected in cell-free samples from a subject, for example, bodily fluid samples from a subject, such as whole blood, plasma, or serum. As such, the present disclosure provides miRNAs that can be used to differentiate between the presence or absence of colon cell proliferative disorder, high-risk or low-risk colorectal lesions that warrant treatment such as, surgical resection, immunotherapy, radiation, or chemotherapy) and low-risk colorectal lesions that can be monitored. Monitoring and confirmation of the presence of colon cell proliferative disorder or lesions can be carried out, for example, by colonoscopy, ultrasound, MM, or CT scan.

The present disclosure relates generally to cancer detection and disease monitoring. More particularly, the field relates to cancer-related microRNA (miRNA) detection and disease monitoring in early-stage colorectal cancer. Specifically, circulating miRNA signature panels and uses thereof are provided for identifying human subjects having, or at risk of developing, colon cell proliferative disorders such as colorectal cancer (CRC) and/or colorectal adenomas (CA), for example, advanced colorectal adenomas (AA).

The present disclosure relates generally to cancer detection and disease monitoring. The present disclosure identifies miRNAs in a subject that are indicative of the presence of a colon cell proliferative disorder, or a high risk of developing a colon cell proliferative disorder, for example, when the subject has a colorectal lesion. Cancer screening and monitoring improve survival outcomes because early detection enables elimination of the cancer before its growth and spread. In colorectal cancer, for instance, colonoscopies play a role in improving early diagnosis. Unfortunately, patient compliance rates are low, and screening is conducted below recommended regularity due to the invasiveness of the procedure.

Described herein are methods for screening or identifying subjects at risk of suffering from a colon cell proliferative disorder based on an expression profile or abundance of miRNAs that are up-regulated or over-expressed in subjects suffering from said diseases. Further described herein are methods for obtaining data useful for diagnosis of a colon cell proliferative disorder in a subject, for example, a human subject. Non-limiting examples of a colon cell proliferative disorder include colorectal cancer, colorectal adenoma, or advanced colorectal adenoma.

As used herein, the term “high risk of developing a colon cell proliferative disorder” refers to a subject having an increased risk of developing a colon cell proliferative disorder in the near future as compared to a subject not having the colon cell proliferative disorder or having a low risk of developing the colon cell proliferative disorder in the near future. As used herein, the term “near future” refers to a duration of about 1 month to about 2 years, about 6 months to about 18 months, or about 1 year.

A colon cell proliferative disorder may be of any tumor stage (e.g., TX, T0, Tis, T1, T2, T3, T4); any regional lymph node or distant metastasis stage (e.g., NX, N0, N1, M0, M1); any stage (e.g., Stage 0 (Tis, N0, M0), Stage IA (T1, N0, M0), Stage IIA (T3, N0, M0), Stage IIB (T1-3, N1, M0), Stage III (T4, Any N, M0), or Stage IV (Any T, Any N, M1)); resectable; locally advanced (unresectable); or metastatic.

Current screening tools may face challenges due to false positive and false negative results, and less than ideal specificity and sensitivity. An ideal cancer screening tool may have a high Positive Predictive Value (PPV), which minimizes unnecessary investigations (low false positives) but detects a vast majority of cancers (low false negative). Another key compromise is “detection sensitivity”, as distinct from test sensitivity, which generally refers to the lower limit of detecting a tumor based on size. Allowing a tumor to grow to a size large enough to release circulating tumor markers at detectable levels defeats the purpose of early detection and prevention of cancer progression. Hence, the present disclosure addresses a need for highly sensitive and effective blood-based screens for early diagnosis of colorectal cancer.

The detection of circulating tumor DNA, also referred to as a “liquid biopsy,” may enable the detection and informative investigation of tumors in a non-invasive manner. Identification of tumor specific mutations in these liquid biopsies may be used to diagnose colon, breast, and prostate cancers. However, due to the high background of normal (e.g., non-tumor-derived) DNA present in circulation, these techniques may be limited in sensitivity. Thus, there remains a need for more sensitive and specific screening tools for detecting early-stage or low tumor-burden colorectal cancer tumor markers for relapse screening and primary screening of at-risk populations.

The present disclosure provides methods and systems directed to profiling circulating miRNAs associated with a colon cell proliferative disorder and progression thereof, for example, a colorectal cancer. Those miRNAs that are indicative of the presence of a colon cell proliferative disorder or a high risk of developing the colon cell proliferative disorder may be used for diagnosing, treating, or preventing progression of a colon cell proliferative disorders as early as possible, for example, when a subject only has a colorectal lesion. Further provided herein are kits and methods for diagnosing colon cell proliferative disorders or assessing the risk of developing colon cell proliferative disorders in a subject, particularly, when the subject has a colorectal lesion.

MiRNAs generally refer to small non-coding RNAs of approximately 18-22 nucleotides found in plants and animals. MiRNAs may post-transcriptionally regulate mRNA targets by binding to a specific site in the 3′-untranslated regions (3′-UTRs), thereby promoting degradation or inhibiting translation of these mRNA targets. MiRNAs may contribute to multiple physiological cellular functions such as proliferation, differentiation, and apoptosis. Dysregulation of miRNAs may play a crucial role in cancer, as miRNAs regulate the expression of oncogenes and tumor suppressor genes. Cell-free miRNA (cfmiRNA), circulating tumor cells (CTCs), circulating tumor DNA (ctDNA), tumor-educated platelets (TEPs), and extracellular vesicles (EVs) may aid in the detection of disease states, and provide relevant prognostic and predictive information about disease status.

Encoded by eukaryotic nuclear DNA, miRNAs may function via base-pairing with complementary sequences within mRNA molecules, usually resulting in gene silencing via translational repression or target degradation. miRNAs are transcribed by RNA polymerase II as large RNA precursors called pri-miRNAs. Pri-miRNAs may be processed further in the nucleus to produce pre-miRNAs. Pre-miRNAs may be about 70-nucleotides in length and are folded into imperfect stem-loop, or “hairpin” structures. Pre-miRNAs may be then exported into the cytoplasm and undergo additional processing to generate mature miRNAs. An miRNA profile of a sample may indicate expression levels of various miRNAs in the sample.

A differentially expressed miRNA may be a miRNA that is either over-expressed, up-regulated, under-expressed, or down-regulated in a sample relative to expression levels in a reference sample (e.g., test cell of a tissue sample compared to a control cell, or a cellular or acellular fluid sample, or a reference expression level (a reference value)). A reference expression level may reflect that of a “normal” state (e.g., lacking the disease) or the corresponding diseased state of interest in a relevant population (e.g., an epidemiologically relevant population), for example.

In some embodiments, a miRNA is identified as “differentially expressed” or “differentially abundant” if the miRNA is expressed in a sample by at least about 1.8-fold higher or lower than the corresponding miRNA in a control sample or reference expression level, or the difference in the expression level between the sample and the control sample or reference expression level has statistical significance (p value) of less than 0.05.

In some embodiments, a miRNA is identified as a “differentially expressed” or “differentially abundant” if the miRNA is expressed in the sample at about 2-fold, about 3-fold, about 4-fold, about 5-fold, or greater than 5-fold higher or lower than the corresponding miRNA in the control sample or reference expression sample. In some embodiments, expression levels are normalized based on a reference standard such as, but not limited to, log2, counts per million, normalized to synthetic spike-ins, or normalized to housekeeping genes.

A differentially expressed miRNA may be a miRNA which is either present in a sample, but rarely observed in reference samples, or absent in a sample but commonly found in reference amples (e.g., test cell of a tissue sample compared to a control cell, or a cellular or acellular fluid sample, or a reference expression level (a reference value)).

In an aspect, provided herein are methods that use a panel of miRNAs useful for distinguishing samples from subjects based on a disease status. In other aspects, provided herein are methods, assays, and kits directed to detecting, differentiating, and distinguishing a colon cell proliferative disorder using a panel of miRNAs. Non-limiting examples of colon cell proliferative disorder include adenocarcinomas, adenomas, polyps, squamous cell cancers, carcinoid tumors, sarcomas, and lymphomas.

In some embodiments, the method comprises the use of one or more miRNAs selected as markers for the differentiation, detection, and distinguishing of a colon cell proliferative disorder.

### I. Definitions

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject can be a person that has cancer or is suspected of having cancer. The subject may be displaying a symptom indicative of a health, physiological state, or condition of the subject, such as a cancer or other disease, disorder, or condition of the subject. As an alternative, the subject can be asymptomatic with respect to such health or physiological state or condition.

As used herein, the term “sample” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck RNA Complete BCT), or a cell-free DNA collection tube (e.g., Streck Cell-Free DNA BCT). Cell-free biological samples may be derived from whole blood samples by fractionation (e.g., by differential centrifugation). Biological samples or derivatives thereof may contain cells. For example, a biological sample may be a blood sample or a derivative thereof (e.g., blood collected by a collection tube or blood drops).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a population of nucleic acid molecules having a nucleotide sequence whose presence, amount, or sequence, or changes thereof, is/are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

The terms “cell-free nucleic acid” or “cfNA”, as used herein, generally refer to nucleic acids in a biological sample that are not contained in a cell. Non-limiting examples of cfNA include cell-free RNA (cfRNA) and cell-free DNA (cfDNA). cfNA may circulate freely in a bodily fluid, such as in the bloodstream.

As used herein, the term “cell-free sample” generally refers to a biological sample that is substantially devoid of intact cells. A cell-free sample may be derived from a biological sample that is itself substantially devoid of cells or may be derived from a sample from which cells have been removed. Non-limiting examples of cell-free samples include those derived from blood, serum, plasma, urine, semen, sputum, feces, ductal exudate, lymph, and recovered lavage.

As used herein, the term “circulating tumor DNA” or “ctDNA” generally refers to cfDNA originating from a tumor.

As used herein, the term “colon cell proliferative disorder” generally refers to a disorder or disease that comprises disordered or aberrant proliferation of cells in the colon or rectum. Non-limiting examples of colon cell proliferative disorders include adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

As used herein, the term “healthy” generally refers to subject not having a colorectal cell proliferation disorder. While health is a dynamic state, as used herein, the term refers to the pathological state of a subject lacking a disease state that reference is being made to in a particular statement. In an example, when referring to a signature panel capable of classifying subjects with colorectal cancer, a healthy individual, a healthy sample, or sample from a healthy individual refers to an individual lacking colorectal cancer (CRC), advanced adenoma (AA), or benign adenoma (NAA). While other diseases or states of health may be present in that subject, as used herein, the term “healthy” indicates the lack of a stated disease for comparison or classification purposes between subjects having and lacking a disease state being discussed.

The term “minimal residual disease” or “MRD” generally refers to the small number of cancer cells in the body of a subject after cancer treatment. MRD testing may be performed to determine effectiveness of a cancer treatment and to guide further treatment plans.

As used herein, the term “screening” generally refers to examination or testing of a population of subjects at risk of suffering from a colorectal cancer or colorectal adenoma, with the objective of discriminating healthy subjects from subjects who are suffering from an undiagnosed colorectal cancer or colorectal adenoma or subjects at high risk of suffering from said indications.

As used herein, the term “colorectal cancer” generally refers to a medical condition characterized by cancer of cells of the intestinal tract below the small intestine (e.g., the large intestine (colon), for example, the cecum, ascending colon, transverse colon, descending colon, sigmoid colon, and rectum).

As used herein, the term “colorectal adenoma” generally refers to adenomas of the colon, also called adenomatous polyps, which is a benign and pre-cancerous stage of the colorectal cancer. Colorectal adenomas may be indicative of a high risk of progression to colorectal cancer.

As used herein, the term “advanced colorectal adenoma” generally refers to adenomas having a size of at least 10 mm or histologically having high grade dysplasia or a villous component higher than 20%.

As used herein, the terms “minimally-invasive biological sample” or “non-invasive sample” generally refer to any sample which is taken from the body of the patient without the need of instruments, other than fine needles used for obtaining blood from a subject. In some embodiments, minimally-invasive biological samples include blood, serum, or plasma samples.

As used herein, the terms “up-regulated” or “over-expressed” generally refer to an increase in an expression level with respect to a given “threshold value” or “cutoff value” by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more than 150%.

As used herein, the terms “threshold value” or “cutoff value”, when referring to the expression levels, generally refer to a reference expression level indicative that a subject is likely to suffer from colorectal cancer or colorectal adenoma with a given sensitivity and specificity if the expression levels of the subject are above said threshold or cut-off or reference levels.

As used herein, the term “kit” is not limited to any specific device and may include any device suitable for implementing systems and methods of the present disclosure such as, but not limited to, microarrays, bioarrays, biochips, or biochip arrays.

As used herein, the terms cancer “type” and “subtype” generally are used relatively herein, such that one “type” of cancer, such as breast cancer, may be “subtypes” based on, e.g., stage, morphology, histology, gene expression, receptor profile, mutation profile, aggressiveness, prognosis, and malignant characteristics. Likewise, “type” and “subtype” may be applied at a finer level, e.g., to differentiate one histological “type” into “subtypes”, e.g., defined according to mutation profile or gene expression. Cancer “stage” is also used to refer to classification of cancer types based on histological and pathological characteristics relating to disease progression.

As used herein, the term “miRNA” or “miR” or “microRNA” generally refers to a non-coding RNA between 17 and 25 nucleobases in length which hybridizes to and regulates the expression of a coding RNA. A 17-25 nucleotide miRNA molecule can be obtained from a miR precursor through natural processing routes (e.g., using intact cells or cell lysates) or by synthetic processing routes (e.g., using isolated processing enzymes, such as isolated Dicer, Argonaut, or RNAase III). The 17-25 nucleotide RNA molecule can also be produced directly by biological or chemical syntheses, without having been processed from a miR precursor.

As used herein, the term “miRNA molecule” generally refers to any nucleic acid molecule representing a miRNA. Non-limiting examples include natural miRNA molecules, pre-miRNA, pri-miRNA, and miRNA molecules that are identical in nucleic acid sequence to the natural forms as well as the nucleic acid sequences of these natural forms in which one or more nucleic acids has been replaced or is represented by one or more DNA nucleotide and/or nucleic acid analogue. In some cases, miRNA molecules are referred to as nucleic acid molecules encoding a miRNA or simply nucleic acid molecule.

As used herein, the term “miRNA profile” generally refers to a collection of expression levels or abundance of a plurality of miRNAs. A miRNA profile is a quantitative measure of individual miRNA expression levels or abundance. Hereby, each miRNA is represented by a numerical value. The higher the value of an individual miRNA the higher is the expression level of this miRNA. A miRNA profile is obtained from the RNA of a biological sample. Non-limiting examples of technologies that may be used to determine a miRNA profile include microarrays, RT-PCR, and Next Generation Sequencing. RNA, total-RNA, or any fraction thereof can be used as a starting material for analysis. The plurality of miRNAs that are determined by a miRNA profile can range from a selection of one up to all known miRNAs.

As used herein, the term “pre-determined set of miRNAs” or “miRNA signature” generally refers to a fixed defined set of miRNAs which is able to differentiate between a condition 1 and another condition 2. For example, condition 1 is colorectal cancer and condition 2 is normal control. In this case, the corresponding pre-determined set of miRNAs is able to differentiate between a samples derived from a colorectal cancer patient or a normal control patient. Alternatively, if condition 1 is colorectal cancer and condition 2 is advanced adenoma, the corresponding pre-determined set of miRNAs is able to differentiate between a colorectal cancer patient and an advanced adenoma patient. To perform a sample analysis, e.g., on a matrix that may be used to determine a miRNA profile, these fixed defined set of miRNAs are represented by probes or other methods that are defined by the pre-determined set of miRNAs. Methods can be selected for sequencing using targets methods, e.g., transcriptome-wide miRNA sequencing and dd/q-PCR methods. For example, when the pre-determined set of miRNAs for diagnosing colorectal cancer from healthy controls consists of 25 miRNAs, probes or methods capable for detecting these 25 miRNAs have to be implemented for performing the diagnostic analysis.

As used herein, the term “common miRNA signature profile” generally refers to a non-fixed defined set of miRNAs or non-coding RNAs which is able to differentiate between a condition 1 and another condition 2. The common miRNA or non-coding RNA signature profile is calculated on the fly from a plurality of miRNA profiles that are stored, e.g. in a database. The common miRNA signature profile that is able to differentiate between a condition and another condition 2 changes as soon as a new profile is added to the database which is relevant to either to state of health 1 or another condition 2. In this respect, a common miRNA signature profile is different from a pre-determined set of miRNAs. Furthermore, the basis for generating the common miRNA signature profile, e.g., the miRNA profiles stored in the database, is generated from capture probes, e.g. on a matrix that is representing as much as possible different capture probes for detecting as many miRNAs as possible.

As used herein, the terms “non-coding RNA” or “ncRNA” generally refer to a functional RNA molecule that is not translated into a protein. In some cases, ncRNA are refers to as non-protein-coding RNA (npcRNA), non-messenger RNA (nmRNA), small non-messenger RNA (snmRNA), or functional RNA (fRNA). The term small RNA (sRNA) is often used for bacterial ncRNAs. The DNA sequence from which a non-coding RNA is transcribed as the end product is often called an RNA gene or non-coding RNA gene. Non-coding RNA genes include highly abundant and functionally important RNAs such as transfer RNA (tRNA) and ribosomal RNA (rRNA), as well as RNAs such as snoRNAs, microRNAs, siRNAs, and piRNAs and long ncRNAs, such as Xist and HOTAIR. The number of ncRNAs encoded within the human genome is unknown. However, recent transcriptomic and bioinformatic studies suggest the existence of thousands of ncRNAs. Since most of the newly identified ncRNAs have not been validated for function, many ncRNAs may be non-functional.

### II. Assaying Samples

The cell-free biological samples may be obtained or derived from a human subject. The cell-free biological samples may be stored in a variety of storage conditions before processing, such as different temperatures (e.g., at room temperature, under refrigeration or freezer conditions, e.g., at 25° C., at 4° C., at −18° C., −20° C., or at −80° C.) or different suspensions (e.g., EDTA collection tubes, cell-free RNA collection tubes, or cell-free DNA collection tubes).

The cell-free biological sample may be obtained from a subject with a cancer, a subject that is suspected of having a cancer, or a subject that does not have or is not suspected of having the cancer.

The cell-free biological sample may be obtained before and/or after treatment of a subject with the cancer. Cell-free biological samples may be obtained from a subject during a treatment or a treatment regime. Multiple cell-free biological samples may be obtained from a subject to monitor the effects of the treatment over time. The cell-free biological sample may be taken from a subject known or suspected of having a cancer for which a definitive positive or negative diagnosis is not available via clinical tests. The sample may be taken from a subject suspected of having cancer. The cell-free biological sample may be taken from a subject experiencing unexplained symptoms, such as fatigue, nausea, weight loss, aches and pains, weakness, or bleeding. The cell-free biological sample may be taken from a subject having explained symptoms. The cell-free biological sample may be taken from a subject at risk of developing a cancer due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The cell-free biological sample may contain one or more analytes capable of being assayed, such as cell-free ribonucleic acid (cfRNA) molecules suitable for assaying to generate transcriptomic data, cell-free deoxyribonucleic acid (cfDNA) molecules suitable for assaying to generate genomic data, or a mixture or combination thereof. One or more such analytes (e.g., cfRNA molecules and/or cfDNA molecules) may be isolated or extracted from one or more cell-free biological samples of a subject for downstream assaying using one or more suitable assays.

After obtaining a cell-free biological sample from the subject, the cell-free biological sample may be processed to generate datasets indicative of a cancer of the subject. For example, a presence, absence, or quantitative assessment of nucleic acid molecules of the cell-free biological sample at a panel of cancer-associated genomic loci (e.g., quantitative measures of RNA transcripts or DNA at the cancer-associated genomic loci). Processing the cell-free biological sample obtained from the subject may include: (i) subjecting the cell-free biological sample to conditions that are sufficient to isolate, enrich, or extract a plurality of nucleic acid molecules, and (ii) assaying the plurality of nucleic acid molecules to generate the dataset.

In some embodiments, a plurality of nucleic acid molecules is extracted from the cell-free biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a MagMAX mirVana Total RNA Isolation Kit, a QIAamp ccfDNA/RNA Kit, a Zymo Quick-cfRNA Serum & Plasma Kit, a FastDNA Kit protocol from MP Biomedicals, a QIAamp DNA cell-free biological minikit from Qiagen, or a cell-free biological DNA isolation kit from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification, e.g., of RNA or DNA molecules. In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with cancers. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a cell-free biological sample may be tagged, e.g., with identifiable tags, to enable multiplexing of a plurality of samples. Any number of RNA or DNA samples may be multiplexed. For example, a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the cancer. For example, the sequence reads may be aligned to one or more reference genomes (e.g., a genome of one or more species such as a human genome). The aligned sequence reads may be quantified at one or more genomic loci to generate the datasets indicative of the cancer. For example, quantification of sequences corresponding to a plurality of genomic loci associated with cancers may generate the datasets indicative of the cancer.

The assay readouts may be quantified at one or more genomic loci (e.g., cancer-associated genomic loci) to generate the data indicative of the cancer. For example, quantification of array hybridization or polymerase chain reaction (PCR) corresponding to a plurality of genomic loci (e.g., cancer-associated genomic loci) may generate data indicative of the cancer. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays may be used to simultaneously process cell-free biological samples of a subject. For example, a first assay may be used to process a first cell-free biological sample obtained or derived from the subject to generate a first dataset indicative of the cancer; and a second assay different from the first assay may be used to process a second cell-free biological sample obtained or derived from the subject to generate a second dataset indicative of the cancer. Any or all of the first dataset and the second dataset may then be analyzed to assess the cancer of the subject. For example, a single diagnostic index or diagnosis score can be generated based on a combination of the first dataset and the second dataset. As another example, separate diagnostic indexes or diagnosis scores can be generated based on the first dataset and the second dataset.

### III. Signature Panels

The present disclosure provides methods and systems to analyze biological samples to obtain measurable features from a combination of miRNA molecules identified in the sample that are associated with the development of a colon cell proliferative disorder. The collection of identified miRNA molecules may form a signature panel where the signature is characteristic of a colon cell proliferative disorder or a stage thereof. The features from the signature panel may be processed using a trained algorithm (e.g., a machine learning model) to create a classifier configured to stratify a population of subjects as having a colon cell proliferative disorder or not having a colon cell proliferative disorder. The methods may be characterized by using one or more nucleic acids having miRNAs described in the signature panels.

The miRNA signature panels described herein may enable rapid and specific analysis of specific miRNAs associated with colon cell proliferative disorders. The signature panels as described and employed in the methods herein may be used for the improved diagnosis, prognosis, treatment selection, and monitoring (e.g., treatment monitoring) of colon cell proliferative disorders.

The signature panels and methods provide significant improvements over current approaches in that there is a need for markers or signature panels used to detect early-stage colon cell proliferative disorders from bodily fluid samples such as whole blood, plasma, or serum. Current methods used to detect and diagnose colon cell proliferative disorders may include colonoscopy, sigmoidoscopy, and fecal occult blood colon cancer. In comparison to these methods, the methods provided herein may be much less invasive than colonoscopy, and equally, if not more sensitive, than sigmoidoscopy, fecal immunochemical test (FIT), and fecal occult blood test (FOBT). Compared to the current use of these markers, methods provided herein may provide significant advantages in terms of sensitivity and specificity due to the advantageous combination of using a gene panel and highly sensitive assay techniques.

A signature panel comprising informative miRNAs may be selected according to the purpose of the intended assay. For targeted methods, primer pairs may be designed based on the set of intended target miRNAs. In some embodiments, the set of miRNAs comprises at least one, at least two, at least three, or more than three miRNAs selected from the group listed in TABLE 1. In some embodiments, the set of miRNAs comprise all the regions listed in TABLE 1. In some embodiments, the set of miRNAs associated with colorectal cancer is selected from the group listed in TABLE 1.

TABLE 1

| | | | | |
|---|---|---|---|---|
| hsa-mir-889 | hsa-mir-6511a-3 | hsa-mir-5481 | hsa-mir-4484 | hsa-mir-320c-1 |
| hsa-mir-543 | hsa-mir-376a-2 | hsa-mir-548v | hsa-mir-4521 | hsa-mir-320d-1 |
| hsa-mir-376b | hsa-mir-155 | hsa-mir-106b | hsa-mir-4706 | hsa-mir-320d-2 |
| hsa-mir-335 | hsa-mir-3140 | hsa-mir-133b | hsa-mir-487a | hsa-mir-324 |
| hsa-mir-1185-1 | hsa-mir-1277 | hsa-mir-548h-2 | hsa-mir-548ac | hsa-mir-331 |
| hsa-mir-548k | hsa-mir-340 | hsa-mir-136 | hsa-mir-5588 | hsa-mir-3529 |
| hsa-mir-12135 | hsa-mir-548n | hsa-mir-26b | hsa-mir-579 | hsa-mir-374c |
| hsa-mir-369 | hsa-mir-518b | hsa-mir-433 | hsa-mir-6734 | hsa-mir-431 |
| hsa-mir-190a | hsa-mir-654 | hsa-mir-4719 | hsa-mir-6882 | hsa-mir-450b |
| hsa-mir-6770-1 | hsa-mir-5581 | hsa-mir-3610 | hsa-mir-9-1 | hsa-mir-4651 |
| hsa-mir-382 | hsa-mir-409 | hsa-mir-374b | hsa-mir-93 | hsa-mir-4716 |
| hsa-mir-1843 | hsa-mir-628 | hsa-mir-376c | hsa-let-7i | hsa-mir-4763 |
| hsa-mir-142 | hsa-mir-10399 | hsa-mir-4779 | hsa-mir-103b-1 | hsa-mir-4772 |
| hsa-mir-485 | hsa-mir-3184 | hsa-mir-496 | hsa-mir-103b-2 | hsa-mir-548a-1 |
| hsa-mir-548ax | hsa-mir-423 | hsa-mir-622 | hsa-mir-12136 | hsa-mir-548ah |
| hsa-mir-548e | hsa-mir-548z | hsa-mir-671 | hsa-mir-1283-2 | hsa-mir-548aj-1 |
| hsa-mir-548al | hsa-mir-374a | hsa-mir-6876 | hsa-mir-1287 | hsa-mir-5703 |
| hsa-mir-548am | hsa-mir-548a-3 | hsa-let-7b | hsa-mir-130a | hsa-mir-574 |
| hsa-mir-590 | hsa-mir-6770-2 | hsa-mir-103a-1 | hsa-mir-146a | hsa-mir-6729 |
| hsa-mir-135a-2 | hsa-mir-1185-2 | hsa-mir-103a-2 | hsa-mir-146b | hsa-mir-6781 |
| hsa-mir-6770-3 | hsa-mir-6077 | hsa-mir-10400 | hsa-mir-151b | hsa-mir-6802 |
| hsa-mir-410 | hsa-mir-3202-1 | hsa-mir-135a-1 | hsa-mir-154 | hsa-mir-7-1 |
| hsa-mir-376a-1 | hsa-mir-548o-2 | hsa-mir-139 | hsa-mir-18a | hsa-mir-7111 |
| hsa-mir-377 | hsa-mir-3143 | hsa-mir-191 | hsa-mir-2278 | hsa-mir-9902-1 |
| hsa-mir-570 | hsa-mir-5009 | hsa-mir-2392 | hsa-mir-2355 | |
| hsa-mir-381 | hsa-mir-548g | hsa-mir-26a-2 | hsa-mir-3138 | |
| hsa-mir-665 | hsa-mir-656 | hsa-mir-320c-2 | hsa-mir-3168 | |
| hsa-mir-758 | hsa-mir-6818 | hsa-mir-4468 | hsa-mir-3202-2 | |

In some embodiments, the set of miRNAs associated with colorectal cancer is selected from the group listed in TABLE 2.

TABLE 2

hsa-mir-889
hsa-mir-543
hsa-mir-376b
hsa-mir-335
hsa-mir-1185-1

In some embodiments, the set of miRNAs associated with colorectal cancer is selected from the group listed in TABLE 3.

TABLE 3

| | |
|---|---|
| hsa-mir-889 | hsa-mir-548k |
| hsa-mir-543 | hsa-mir-12135 |
| hsa-mir-376b | hsa-mir-369 |
| hsa-mir-335 | hsa-mir-190a |
| hsa-mir-1185-1 | hsa-mir-6770-1 |

In some embodiments, the set of miRNAs associated with colorectal cancer is selected from the group listed in TABLE 4.

TABLE 4

| | | |
|---|---|---|
| hsa-mir-889 | hsa-mir-548k | hsa-mir-382 |
| hsa-mir-543 | hsa-mir-12135 | hsa-mir-1843 |
| hsa-mir-376b | hsa-mir-369 | hsa-mir-142 |
| hsa-mir-335 | hsa-mir-190a | hsa-mir-485 |
| hsa-mir-1185-1 | hsa-mir-6770-1 | hsa-mir-548ax |

In some embodiments, the set of miRNAs associated with colorectal cancer is selected from the group listed in TABLE 5.

TABLE 5

| | | | |
|---|---|---|---|
| hsa-mir-889 | hsa-mir-548k | hsa-mir-382 | hsa-mir-548e |
| hsa-mir-543 | hsa-mir-12135 | hsa-mir-1843 | hsa-mir-548al |
| hsa-mir-376b | hsa-mir-369 | hsa-mir-142 | hsa-mir-548am |
| hsa-mir-335 | hsa-mir-190a | hsa-mir-485 | hsa-mir-590 |
| hsa-mir-1185-1 | hsa-mir-6770-1 | hsa-mir-548ax | hsa-mir-135a-2 |

A profile of differentially expressed miRNAs represents a set of miRNAs that are differentially expressed in a fluid or tissue sample compared to a control or reference level. The profile of differentially expressed miRNAs includes a profile of down-regulated or under-expressed miRNAs and a profile of up-regulated/over-expressed miRNAs.

In some embodiments, miRNAs are differentially expressed in a sample of a subject having high risk of developing colon cell proliferative disorders as compared to the corresponding sample of a subject having low risk of developing colon cell proliferative disorders. In some embodiments, each of the subjects having high risk of developing colon cell proliferative disorders and the subject having low risk of developing colon cell proliferative disorders have a colorectal lesion. The miRNAs that are differentially expressed in a sample of a subject having high risk of developing or having colon cell proliferative disorders can be used as biomarkers for diagnosis or prevention of colon cell proliferative disorders. For example, miRNAs differentially expressed in a sample of a subject having high risk of developing colon cell proliferative disorders as compared to the corresponding cell of a subject having low risk of developing colon cell proliferative disorders comprises one or more of hsa-mir-889, hsa-mir-543, hsa-mir-376b, hsa-mir-335, hsa-mir-1185-1, hsa-mir-548k, hsa-mir-12135, hsa-mir-369, hsa-mir-190a, hsa-mir-6770-1, hsa-mir-382, hsa-mir-1843, hsa-mir-142, and hsa-mir-485, hsa-mir-548ax, hsa-mir-548e, hsa-mir-548a1, hsa-mir-548am, hsa-mir-590, hsa-mir-135a-2, hsa-mir-6770-3, hsa-mir-410, hsa-mir-376a-1, hsa-mir-377, hsa-mir-570, hsa-mir-381, hsa-mir-665, hsa-mir-758, hsa-mir-6511a-3, hsa-mir-376a-2, hsa-mir-155, hsa-mir-3140, hsa-mir-1277, hsa-mir-340, and hsa-mir-548n, hsa-mir-518b, hsa-mir-654, hsa-mir-5581, hsa-mir-409, hsa-mir-628, hsa-mir-10399, hsa-mir-3184, hsa-mir-423, hsa-mir-548z, hsa-mir-374a, hsa-mir-548a-3, hsa-mir-6770-2, hsa-mir-1185-2, hsa-mir-6077, hsa-mir-3202-1, hsa-mir-548o-2, hsa-mir-3143, hsa-mir-5009, hsa-mir-548g, and hsa-mir-656.

In some embodiments, miRNAs that are higher expressed in a sample of a subject having high risk of developing colon cell proliferative disorders as compared to the corresponding sample of a subject having low risk of developing colon cell proliferative disorders comprises one or more of hsa-mir-889, hsa-mir-543, hsa-mir-376b, hsa-mir-335, hsa-mir-1185-1, hsa-mir-548k, hsa-mir-12135, hsa-mir-369, hsa-mir-190a, hsa-mir-6770-1, hsa-mir-382, hsa-mir-1843, hsa-mir-142, and hsa-mir-485, hsa-mir-548ax, hsa-mir-548e, hsa-mir-548a1, hsa-mir-548am, hsa-mir-590, hsa-mir-135a-2, hsa-mir-6770-3, hsa-mir-410, hsa-mir-376a-1, hsa-mir-377, hsa-mir-570, hsa-mir-381, hsa-mir-665, hsa-mir-758, hsa-mir-6511a-3, hsa-mir-376a-2, hsa-mir-155, hsa-mir-3140, hsa-mir-1277, hsa-mir-340, and hsa-mir-548n, hsa-mir-518b, hsa-mir-654, hsa-mir-5581, hsa-mir-409, hsa-mir-628, hsa-mir-10399, hsa-mir-3184, hsa-mir-423, hsa-mir-548z, hsa-mir-374a, hsa-mir-548a-3, hsa-mir-6770-2, hsa-mir-1185-2, hsa-mir-6077, hsa-mir-3202-1, hsa-mir-548o-2, hsa-mir-3143, hsa-mir-5009, hsa-mir-548g, and hsa-mir-656.

In some embodiments, the colon cell proliferative disorder is advanced adenoma.

In some embodiments, miRNAs that are lower expressed in a sample of a subject having high risk of developing colon cell proliferative disorders as compared to the corresponding sample of a subject having low risk of developing colon cell proliferative disorders comprises one or more of hsa-mir-889, hsa-mir-543, hsa-mir-376b, hsa-mir-335, hsa-mir-1185-1, hsa-mir-548k, hsa-mir-12135, hsa-mir-369, hsa-mir-190a, hsa-mir-6770-1, hsa-mir-382, hsa-mir-1843, hsa-mir-142, and hsa-mir-485, hsa-mir-548ax, hsa-mir-548e, hsa-mir-548a1, hsa-mir-548am, hsa-mir-590, hsa-mir-135a-2, hsa-mir-6770-3, hsa-mir-410, hsa-mir-376a-1, hsa-mir-377, hsa-mir-570, hsa-mir-381, hsa-mir-665, hsa-mir-758, hsa-mir-6511a-3, hsa-mir-376a-2, hsa-mir-155, hsa-mir-3140, hsa-mir-1277, hsa-mir-340, and hsa-mir-548n, hsa-mir-518b, hsa-mir-654, hsa-mir-5581, hsa-mir-409, hsa-mir-628, hsa-mir-10399, hsa-mir-3184, hsa-mir-423, hsa-mir-548z, hsa-mir-374a, hsa-mir-548a-3, hsa-mir-6770-2, hsa-mir-1185-2, hsa-mir-6077, hsa-mir-3202-1, hsa-mir-548o-2, hsa-mir-3143, hsa-mir-5009, hsa-mir-548g, and hsa-mir-656.

In an example, a panel of miRNAs have increased expression in samples from subjects with advanced adenoma relative to samples from healthy subjects without advanced adenoma. In an example, the panel includes two or more miRNAs selected from the group listed in TABLE 6. In other examples, the panel includes 3 or more, 4 or more, 5 or more, or 6 or more of the miRNAs listed.

TABLE 6

| | | | |
|---|---|---|---|
| hsa-mir-12135 | hsa-mir-376a-3p | hsa-mir-485-5p | hsa-mir-654-3p |
| hsa-mir-1277 | hsa-mir-376b | hsa-mir-487b-3p | hsa-mir-889 |
| hsa-mir- 323b-3p | hsa-mir- 376c-3p | hsa-mir- 5096 | |
| hsa-mir- 335 | hsa-mir- 382-5p | hsa-mir- 548 | |
| hsa-mir- 369-5p | hsa-mir- 409-3p | hsa-mir- 5585-3p | |

In another example of a panel of miRNAs having increased expression in samples from subjects with advanced adenoma relative to samples from healthy subjects without advanced adenoma, the panel includes two or more miRNAs selected from the group listed in TABLE 7. In other examples, the panel includes 3 or more, 4 or more, 5 or more, or 6 or more of the miRNAs listed.

In some embodiments, the panel is selected from 5 or more miRNAs comprising: a) hsa-miR-1273a, hsa-miR-17-5p, hsa-miR-20a-3p, hsa-miR-20b-5p; b) hsa-miR-3065-5p, hsa-miR-4785, hsa-miR-5096, hsa-miR-5189-5p, or c) hsa-miR-545-3p, hsa-miR-570-3p, hsa-miR-624-3p, hsa-mir-1181, hsa-mir-6073.

TABLE 7

| | | | |
|---|---|---|---|
| hsa-miR-1273a | hsa-miR-3065-5p | hsa-miR-545-3p | hsa-mir-6073 |
| hsa-miR-17-5p | hsa-miR-4785 | hsa-miR-570-3p | |
| hsa-miR-20a-3p | hsa-miR-5096 | hsa-miR-624-3p | |
| hsa-miR-20b-5p | hsa-miR-5189-5p | hsa-mir-1181 | |

In an example, a panel of miRNAs have decreased expression in samples from subjects with advanced adenoma relative to samples from healthy subjects without advanced adenoma. In an example, the panel includes two or more miRNAs selected from the group listed in TABLE 8. In other examples, the panel includes 3 or more, 4 or more, 5 or more, or 6 or more of the miRNAs listed.

TABLE 8

hsa-mir-34a-5p
hsa-mir-3687
hsa-mir-4492
hsa-mir-455-3p
hsa-mir-4727-3p
hsa-mir-524-5p
hsa-mir-5701

In an example, a panel of miRNAs have increased expression in samples from subjects with colorectal cancer relative to samples from healthy subjects without colorectal cancer. In an example, the panel includes two or more miRNAs selected from the group listed in TABLE 9. In other examples, the panel includes 3 or more, 4 or more, 5 or more, or 6 or more of the miRNAs listed.

TABLE 9

| | | | |
|---|---|---|---|
| hsa-mir- 10a-3p | hsa-mir- 196a-5p | hsa-mir- 3183 | hsa-mir- 6089-2 |
| hsa-mir- 1207 | hsa-mir- 200a-3p | hsa-mir- 3194-5p | hsa-mir- 6749 |
| hsa-mir- 1233-5p | hsa-mir- 200a-5p | hsa-mir- 3683 | hsa-mir- 6760-5p |
| hsa-mir- 1246 | hsa-mir- 200b-3p | hsa-mir- 4436b-1 | hsa-mir- 6846 |
| hsa-mir- 1247-5p | hsa-mir- 200b-5p | hsa-mir- 4484 | hsa-mir- 6867-5p |
| hsa-mir- 1273d | hsa-mir- 200c-3p | hsa-mir- 4670 | hsa-mir- 6873 |
| hsa-mir- 1273e | hsa-mir- 203a-3p | hsa-mir- 4683 | hsa-mir- 7110 |
| hsa-mir- 1273f | hsa-mir- 205-3p | hsa-mir- 4779 | hsa-mir- 718 |
| hsa-mir- 1275 | hsa-mir- 3131 | hsa-mir- 514b-5p | hsa-mir- 7704 |
| hsa-mir- 1290 | hsa-mir- 3132 | hsa-mir- 552-3p | hsa-mir- 7847-3p |
| hsa-mir- 141-3p | hsa-mir- 3141 | hsa-mir- 5692a-2 | hsa-mir- 9902-1 |
| hsa-mir- 1910-3p | hsa-mir- 3180-3p | hsa-mir- 5698 | hsa-mir- 9902-2 |

In another example of a panel of miRNAs having increased expression in samples from subjects with colorectal cancer relative to samples from healthy subjects without colorectal cancer, the panel includes two or more miRNAs selected from the group listed in TABLE 10. In other examples, the panel includes 3 or more, 4 or more, 5 or more, or 6 or more of the miRNAs listed.

In another example, the panel is selected from 5 or more miRNAs comprising: a) hsa-miR-1250-5p, hsa-miR-1255a, hsa-miR-223-3p, hsa-miR-338-3p, hsa-miR-338-5p; b) hsa-miR-424-5p, hsa-miR-424-3p, hsa-miR-450a-5p, hsa-miR-450b-5p, hsa-miR-4772-3p; c) hsa-miR-4772-5p, hsa-miR-625-5p, hsa-miR-7847-3p, hsa-miR-1181, hsa-miR-3651, hsa-mir-6073; d) hsa-mir-6125, hsa-mir-7704, hsa-miR-19b-3p, hsa-miR-19a-3p, hsa-miR-3157-5p; e) hsa-miR-142-3p, hsa-miR-30c-5p, hsa-miR-6741-5p, hsa-miR-590-3p, hsa-miR-4685-5p; f) hsa-miR-3648, hsa-miR-331-3p, hsa-miR-1303, hsa-miR-6790-3p, hsa-miR-6867-5p, hsa-miR-942-5p; g) hsa-miR-378a-3p, hsa-miR-1287-5p, hsa-mir-4785, hsa-miR-324-3p, hsa-miR-550b-2-5p; h) hsa-miR-200c-3p, hsa-miR-200b-3p, hsa-miR-3679-5p, hsa-miR-550a-3-5p, hsa-miR-3187-3p; i) hsa-miR-181b-5p, hsa-miR-3138, hsa-miR-146a-5p, hsa-miR-6721-5p, hsa-miR-23b-3p, hsa-miR-28-5p; j) hsa-miR-320d, hsa-miR-940, hsa-miR-320d-1, hsa-miR-10a-5p, hsa-miR-340-5p; k) hsa-miR-320b, hsa-miR-335-5p, hsa-miR-320c, hsa-miR-501-3p, hsa-miR-548n; or l) hsa-miR-27a-3p, hsa-miR-3065-3p, hsa-miR-548aa@, hsa-miR-584-3p, hsa-miR-22-3p.

TABLE 10

| | | | |
|---|---|---|---|
| hsa-miR-1250-5p | hsa-mir-6125 | hsa-miR-378a-3p | hsa-miR-320d |
| hsa-miR-1255a | hsa-mir-7704 | hsa-miR-1287-5p | hsa-miR-940 |
| hsa-miR-223-3p | hsa-miR-19b-3p | hsa-mir-4785 | hsa-miR-320d-1 |
| hsa-miR-338-3p | hsa-miR-19a-3p | hsa-miR-324-3p | hsa-miR-10a-5p |
| hsa-miR-338-5p | hsa-miR-3157-5p | hsa-miR-550b-2-5p | hsa-miR-340-5p |
| hsa-miR-424-5p | hsa-miR-142-3p | hsa-miR-200c-3p | hsa-miR-320b |
| hsa-miR-424-3p | hsa-miR-30c-5p | hsa-miR-200b-3p | hsa-miR-335-5p |
| hsa-miR-450a-5p | hsa-miR-6741-5p | hsa-miR-3679-5p | hsa-miR-320c |
| hsa-miR-450b-5p | hsa-miR-590-3p | hsa-miR-550a-3-5p | hsa-miR-501-3p |
| hsa-miR-4772-3p | hsa-miR-4685-5p | hsa-miR-3187-3p | hsa-miR-548n |
| hsa-miR-4772-5p | hsa-miR-3648 | hsa-miR-181b-5p | hsa-miR-27a-3p |
| hsa-miR-625-5p | hsa-miR-331-3p | hsa-miR-3138 | hsa-miR-3065-3p |
| hsa-miR-7847-3p | hsa-miR-1303 | hsa-miR-146a-5p | hsa-miR-548aa@ |
| hsa-miR-1181 | hsa-miR-6790-3p | hsa-miR-6721-5p | hsa-miR-584-3p |
| hsa-miR-3651 | hsa-miR-6867-5p | hsa-miR-23b-3p | hsa-miR-22-3p |
| hsa-mir-6073 | hsa-miR-942-5p | hsa-miR-28-5p | |

In an example, a panel of miRNAs have decreased expression in samples from subjects with colorectal cancer relative to samples from healthy subjects without colorectal cancer. In an example, the panel includes two or more miRNAs selected from the group listed in TABLE 11.

TABLE 11

hsa-mir-3135b
hsa-mir-581

In some embodiments, the colon cell proliferative disorder is advanced adenoma.

In some embodiments, the colon cell proliferative disorder is colorectal cancer.

In some embodiments, certain specific combinations of biomarkers departing from miR-889 alone, provides better results in terms of frequency in k-fold cross validation, AUC, sensitivity and specificity values for both detecting the presence of advanced colorectal adenoma and for detecting the presence of colorectal cancer in comparison to the use of miR-889, miR-543, miR-376b, miR-335 and miR-1185-1 by themselves.

In some embodiments, such examples include at least (miR-889) or of at least (miR-889 and miR-543) or of at least (miR-889 and miR-376b), or of at least (miR-889 and miR-335), or of at least (miR-889 and miR-1185-1), or of at least (miR-889, miR-543 and miR-376b), or of at least (miR-889, miR-543 and miR-335), or of at least (miR-889, miR-376b and miR-335), or of at least (miR-889, miR-543 and miR-1185-1), or of at least (miR-889, miR-543 and miR-548k), or of at least (miR-889, miR-543 and miR-12135), or of at least (miR-889, miR-543, miR-376b, miR-335 and miR-1185-1) are significantly up-regulated in plasma samples of subjects suffering from colorectal cancer and advanced adenomas. In this sense, as shown in TABLE 1, the combination of at least (miR-889) or of at least (miR-889 and miR-543) or of at least (miR-889 and miR-376b), or of at least (miR-889 and miR-335), or of at least (miR-889 and miR-1185-1), or of at least (miR-889, miR-543 and miR-376b), or of at least (miR-889, miR-543 and miR-335), or of at least (miR-889, miR-376b and miR-335), or of at least (miR-889, miR-543 and miR-1185-1), or of at least (miR-889, miR-543 and miR-548k), or of at least (miR-889, miR-543 and miR-12135), or of at least (miR-889, miR-543, miR-376b, miR-335, and miR-1185-1).

In some embodiments, methods of the present disclosure may comprise administering a treatment to a subject in need thereof (e.g., based on having a colon proliferative disorder). Since colorectal adenoma can be seen as a precursor of colorectal cancer, because of the acknowledged adenoma-carcinoma sequence, and the notion that advanced colorectal adenomas are more likely to transition to cancer, colorectal adenomas (e.g., colorectal advanced adenomas) may be treated, for example, by being removed through colonoscopy (subsequent surveillance may be performed). Treatment of colorectal cancer depends on the stage at which cancer was discovered. Early stage colorectal cancer may be treated with surgery. Approximately 95% of Stage I and 65-80% of Stage II colorectal cancers are curable with surgery. Rectal cancer, however, may require additional radiation therapy to minimize the risk of recurrence. Advanced stage (Stage III and Stage IV) treatment often comprises a combination of therapies, including: surgery, chemotherapy, treatment with antibodies, therapies anti-VEGF/R and radiation. The treatment for a subject having colorectal cancer may be described by, for example, Wolpin et al., “Systemic Treatment of Colorectal Cancer,” Gastroenterology, Volume 134, Issue 5, 2008, Pages 1296-1310.el, ISSN 0016-5085, which is incorporated by reference herein in its entirety.

A treatment may be selected (e.g., from among a plurality of possible treatment options) and administered to the subject based at least in part on a miRNA profile of the subject and/or a set of biological traits of the subject. The biological traits may be a measurement, a diagnosis, a prognosis, or a prediction (e.g., determined using a trained machine learning classifier).

In some embodiments, the biological trait comprises malignancy. [0001] In some embodiments, the biological trait comprises a cancer type. In some embodiments, the biological trait comprises a cancer stage. In some embodiments, the biological trait comprises a cancer classification. In some embodiments, the cancer classification comprises a cancer grade. In some embodiments, the cancer classification comprises a histological classification. In some embodiments, the biological trait comprises a metabolic profile. In some embodiments, the biological trait comprises a mutation. In some embodiments, the mutation is a disease-associated mutation. In some embodiments, the biological trait comprises a clinical outcome. In some embodiments, the biological trait comprises a drug response.

### V. Classifiers, Machine Learning Models & Systems

In some examples, miRNA sequencing features are used as input datasets into trained algorithms (e.g., machine learning models or classifiers) to find correlations between sequence composition and subject groups (e.g., patient groups). Examples of such patient groups include presence or absence of diseases or conditions, elevated or non-elevated risk of diseases or conditions, stages of diseases or conditions, subtypes of diseases or conditions, responders to treatment vs. non-responders to treatment, and progressors versus non-progressors. In some examples, feature matrices are generated to compare samples obtained from subjects with known conditions or characteristics. In some embodiments, samples are obtained from healthy subjects, or subjects who do not have any of the known indications and samples from patients known to have cancer.

As used herein, as it relates to machine learning and pattern recognition, the term “feature” generally refers to an individual measurable property or characteristic of a phenomenon being observed. The concept of “feature” is related to that of an explanatory variable used in statistical techniques such as for example, but not limited to, linear regression and logistic regression. Features may be numeric or categorical (e.g., structural features such as strings and graphs are used in syntactic pattern recognition).

As used herein, the term “input features” (or “features”) generally refers to variables that are used by the trained algorithm (e.g., machine learning model or classifier) to predict an output classification (label) of a sample, e.g., a condition, sequence content (e.g., mutations), suggested data collection operations, or suggested treatments. Values of the variables may be determined for a sample and used to determine a classification.

For a plurality of assays, the system identifies feature sets to input into a trained algorithm (e.g., machine learning model or classifier). The system performs an assay on each biological sample and forms a feature vector from the measured values. The system inputs the feature vector into the machine learning model and obtains an output classification of whether the biological sample has a specified property.

In some embodiments, the machine learning model outputs a classifier capable of distinguishing between two or more groups or classes of subjects or features in a population of subjects or features of the population. In some embodiments, the classifier is a trained machine learning classifier.

In some embodiments, the informative loci or features of biomarkers in a cancer tissue are assayed to form a profile. Receiver-operating characteristic (ROC) curves may be generated by plotting the performance of a particular feature (e.g., any of the biomarkers described herein and/or any item of additional biomedical information) in distinguishing between two populations (e.g., subjects responding and not responding to a therapeutic agent). In some embodiments, the feature data across the entire population (e.g., the cases and controls) are sorted in ascending order based on the value of a single feature.

In some examples, the specified property is selected from healthy vs. cancer, elevated vs. non-elevated risk of disease, disease subtype, disease stage, progressor vs. non-progressor, and responder vs. non-responder.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

### A. Data Analysis

In some examples, the present disclosure provides a system, method, or kit having data analysis realized in software application, computing hardware, or both. In some examples, the analysis application or system comprises at least a data receiving module, a data pre-processing module, a data analysis module (which can operate on one or more types of genomic data), a data interpretation module, or a data visualization module. In some embodiments, the data receiving module can comprise computer systems that connect laboratory hardware or instrumentation with computer systems that process laboratory data. In some embodiments, the data pre-processing module can comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

In some examples, machine learning methods are applied to distinguish samples in a population of samples. In some embodiments, machine learning methods are applied to distinguish samples between healthy and advanced disease (e.g., adenoma) samples.

In some embodiments, the one or more machine learning operations used to train the prediction engine include one or more of: a generalized linear model, a generalized additive model, a non-parametric regression operation, a random forest classifier, a spatial regression operation, a Bayesian regression model, a time series analysis, a Bayesian network, a Gaussian network, a decision tree learning operation, an artificial neural network, a recurrent neural network, a reinforcement learning operation, linear or non-linear regression operations, a support vector machine, a clustering operation, and a genetic algorithm operation.

In some examples, computer processing methods are selected from logistic regression, multiple linear regression (MLR), dimension reduction, partial least squares (PLS) regression, principal component regression, autoencoders, variational autoencoders, singular value decomposition, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, and artificial neural networks.

In some examples, the methods disclosed herein can include computational analysis on nucleic acid sequencing data of samples from a subject or from a plurality of subjects.

### B. Classifier Generation

In an aspect, the disclosed systems and methods provide a classifier generated based on feature information derived from miRNA sequence analysis from biological samples of cfRNA. The classifier forms part of a predictive engine for distinguishing groups in a population based on sequence features identified in biological samples such as cfDNA.

In some embodiments, a classifier is created by normalizing the sequence information by formatting similar portions of the sequence information into a unified format and a unified scale; storing the normalized sequence information in a columnar database; training a prediction engine by applying one or more one machine learning operations to the stored normalized sequence information, the prediction engine mapping, for a particular population, a combination of one or more features; applying the prediction engine to the accessed field information to identify a subject associated with a group; and classifying the subject into a group.

Specificity, as used herein, generally refers to “the probability of a negative test among those who are free from the disease”. It may be calculated by the number of disease-free persons who tested negative divided by the total number of disease-free subjects.

In some examples, the model, classifier, or predictive test has a specificity of at least 40%, at least 45%, at least 50%, at least 55%, at least 60%, at least 65%, at least 70%, at least 75%, at least 80%, at least 85%, at least 90%, at least 95%, or at least 99%.

Sensitivity, as used herein, generally refers to “the probability of a positive test among those who have the disease”. It may be calculated by the number of diseased subjects who tested positive divided by the total number of diseased subjects.

In some examples, the model, classifier, or predictive test has a sensitivity of at least 40%, at least 45%, at least 50%, at least 55%, at least 60%, at least 65%, at least 70%, at least 75%, at least 80%, at least 85%, at least 90%, at least 95%, or at least 99%.

### C. Digital Processing Device

In some examples, the subject matter described herein can include a digital processing device or use of the same. In some examples, the digital processing device can include one or more hardware central processing units (CPU), graphics processing units (GPU), or tensor processing units (TPU) that carry out the device's functions. In some examples, the digital processing device can include an operating system configured to perform executable instructions.

In some examples, the digital processing device can optionally be connected to a computer network. In some examples, the digital processing device may be optionally connected to the Internet. In some examples, the digital processing device may be optionally connected to a cloud computing infrastructure. In some examples, the digital processing device may be optionally connected to an intranet. In some examples, the digital processing device may be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations.

In some examples, the digital processing device can include an operating system configured to perform executable instructions. For example, the operating system can include software, including programs and data, which manages the device's hardware and provides services for execution of applications. Non-limiting examples of operating systems include Ubuntu, FreeB SD, OpenBSD, NetB SD®, Linux, Apple® Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some examples, the operating system may be provided by cloud computing, and cloud computing resources may be provided by one or more service providers.

In some examples, the device can include a storage and/or memory device. The storage and/or memory device may be one or more physical apparatuses used to store data or programs on a temporary or permanent basis. In some examples, the device may be volatile memory and require power to maintain stored information. In some examples, the device may be non-volatile memory and retain stored information when the digital processing device is not powered. In some examples, the non-volatile memory can include flash memory. In some examples, the non-volatile memory can include dynamic random-access memory (DRAM). In some examples, the non-volatile memory can include ferroelectric random access memory (FRAM). In some examples, the non-volatile memory can include phase-change random access memory (PRAM).

In some examples, the device may be a storage device including, for example, CD-ROMs, DVDs, flash memory devices, magnetic disk drives, magnetic tapes drives, optical disk drives, and cloud computing-based storage. In some examples, the storage and/or memory device may be a combination of devices such as those disclosed herein. In some examples, the digital processing device can include a display to send visual information to a user. In some examples, the display may be a cathode ray tube (CRT). In some examples, the display may be a liquid crystal display (LCD). In some examples, the display may be a thin film transistor liquid crystal display (TFT-LCD). In some examples, the display may be an organic light emitting diode (OLED) display. In some examples, an OLED display may be a passive-matrix OLED (PMOLED) or active-matrix OLED (AMOLED) display. In some examples, the display may be a plasma display. In some examples, the display may be a video projector. In some examples, the display may be a combination of devices such as those disclosed herein.

In some examples, the digital processing device can include an input device to receive information from a user. In some examples, the input device may be a keyboard. In some examples, the input device may be a pointing device including, for example, a mouse, trackball, track pad, joystick, game controller, or stylus. In some examples, the input device may be a touch screen or a multi-touch screen. In some examples, the input device may be a microphone to capture voice or other sound input. In some examples, the input device may be a video camera to capture motion or visual input. In some examples, the input device may be a combination of devices such as those disclosed herein.

### D. Non-Transitory Computer-Readable Storage Medium

In some examples, the subject matter disclosed herein can include one or more non-transitory computer-readable storage media encoded with a program including instructions executable by the operating system of an optionally networked digital processing device. In some examples, a computer-readable storage medium may be a tangible component of a digital processing device. In some examples, a computer-readable storage medium may be optionally removable from a digital processing device. In some examples, a computer-readable storage medium can include, for example, CD-ROMs, DVDs, flash memory devices, solid state memory, magnetic disk drives, magnetic tape drives, optical disk drives, cloud computing systems and services, and the like. In some examples, the program and instructions may be permanently, substantially permanently, semi-permanently, or non-transitorily encoded on the media.

### E. Computer Systems

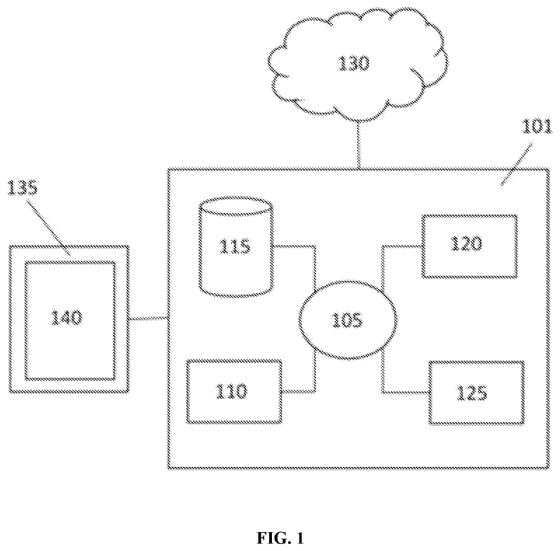

The present disclosure provides computer systems that are programmed to implement methods described herein. FIG. 1 shows a computer system 101 that is programmed or otherwise configured to store, process, identify, or interpret patient data, biological data, biological sequences, and reference sequences. The computer system 101 can process various aspects of patient data, biological data, biological sequences, or reference sequences of the present disclosure. The computer system 101 may be an electronic device of a user or a computer system that is remotely located with respect to the electronic device. The electronic device may be a mobile electronic device.

The computer system 101 comprises a central processing unit (CPU, also “processor” and “computer processor” herein) 105, which may be a single core or multi core processor, or a plurality of processors for parallel processing. The computer system 101 also comprises memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 may be a data storage unit (or data repository) for storing data. The computer system 101 may be operatively coupled to a computer network (“network”) 130 with the aid of the communication interface 120. The network 130 may be the Internet, an internet and/or extranet, or an intranet and/or extranet that is in communication with the Internet. The network 130 in some examples is a telecommunication and/or data network. The network 130 can include one or more computer servers, which can enable distributed computing, such as cloud computing. The network 130, in some examples with the aid of the computer system 101, can implement a peer-to-peer network, which may enable devices coupled to the computer system 101 to behave as a client or a server.

The CPU 105 can execute a sequence of machine-readable instructions, which may be embodied in a program or software. The instructions may be stored in a memory location, such as the memory 110. The instructions may be directed to the CPU 105, which can subsequently program or otherwise configure the CPU 105 to implement methods of the present disclosure. Examples of operations performed by the CPU 105 can include fetch, decode, execute, and writeback.

The CPU 105 may be part of a circuit, such as an integrated circuit. One or more other components of the system 101 may be included in the circuit. In some examples, the circuit is an application specific integrated circuit (ASIC).

The storage unit 115 can store files, such as drivers, libraries and saved programs. The storage unit 115 can store user data, e.g., user preferences and user programs. The computer system 101 in some examples can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein may be implemented by way of machine (e.g., computer processor) executable code stored on an electronic storage location of the computer system 101, such as, for example, on the memory 110 or electronic storage unit 115. The machine-executable or machine-readable code may be provided in the form of software. During use, the code may be executed by the processor 105. In some examples, the code may be retrieved from the storage unit 115 and stored on the memory 110 for ready access by the processor 105. In some examples, the electronic storage unit 115 may be precluded, and machine-executable instructions are stored on memory 110.

The code may be pre-compiled and configured for use with a machine having a processer adapted to execute the code or may be interpreted or compiled during runtime. The code may be supplied in a programming language that may be selected to enable the code to execute in a pre-compiled, interpreted, or as-compiled fashion.

Aspects of the systems and methods provided herein, such as the computer system 101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements comprises optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a nucleic acid sequence, an enriched nucleic acid sample, a miRNA profile, an expression profile, and an analysis of a RNA expression profile. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 105. The algorithm can, for example, store, process, identify, or interpret patient data, biological data, biological sequences, and reference sequences.

In some examples, the subject matter disclosed herein can include at least one computer program or use of the same. A computer program can be a sequence of instructions, executable in the digital processing device's CPU, GPU, or TPU, written to perform a specified task. Computer-readable instructions may be implemented as program modules, such as functions, objects, Application Programming Interfaces (APIs), data structures, and the like, that perform particular tasks or implement particular abstract data types. In light of the disclosure provided herein, a computer program may be written in various versions of various languages.

The functionality of the computer-readable instructions may be combined or distributed as desired in various environments. In some examples, a computer program can include one sequence of instructions. In some examples, a computer program can include a plurality of sequences of instructions. In some examples, a computer program may be provided from one location. In some examples, a computer program may be provided from a plurality of locations. In some examples, a computer program can include one or more software modules. In some examples, a computer program can include, in part or in whole, one or more web applications, one or more mobile applications, one or more standalone applications, one or more web browser plug-ins, extensions, add-ins, or add-ons, or combinations thereof.

In some examples, the computer processing may be a method of statistics, mathematics, biology, or any combination thereof. In some examples, the computer processing method comprises a dimension reduction method including, for example, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, and neural network.

In some examples, the computer processing method is a supervised machine learning method including, for example, a regression, support vector machine, tree-based method, and network.

In some examples, the computer processing method is an unsupervised machine learning method including, for example, clustering, network, principal component analysis, and matrix factorization.

### F. Databases

In some examples, the subject matter disclosed herein can include one or more databases, or use of the same to store patient data, biological data, biological sequences, or reference sequences. Reference sequences may be derived from a database. In view of the disclosure provided herein, many databases may be suitable for storage and retrieval of the sequence information. In some examples, suitable databases can include, for example, relational databases, non-relational databases, object-oriented databases, object databases, entity-relationship model databases, associative databases, and XML databases. In some examples, a database may be internet-based. In some examples, a database may be web-based. In some examples, a database may be cloud computing-based. In some examples, a database may be based on one or more local computer storage devices.

In an aspect, the present disclosure provides a non-transitory computer-readable medium comprising instructions that direct a processor to carry out a method disclosed herein.

In an aspect, the present disclosure provides a computing device comprising the computer-readable medium.

In another aspect, the present disclosure provides a system for performing classifications of biological samples comprising:
a) a receiver to receive a plurality of training samples, each of the plurality of training samples having a plurality of classes of molecules, wherein each of the plurality of training samples comprises one or more known labels
b) a feature module to identify a set of features corresponding to an assay that are operable to be input to the machine learning model for each of the plurality of training samples, wherein the set of features correspond to properties of molecules in the plurality of training samples, wherein for each of the plurality of training samples, the system is operable to subject a plurality of classes of molecules in the training sample to a plurality of different assays to obtain sets of measured values, wherein each set of measured values is from one assay applied to a class of molecules in the training sample, wherein a plurality of sets of measured values are obtained for the plurality of training samples,
c) an analysis module to analyze the sets of measured values to obtain a training vector for the training sample, wherein the training vector comprises feature values of the N set of features of the corresponding assay, each feature value corresponding to a feature and including one or more measured values, wherein the training vector is formed using at least one feature from at least two of the N sets of features corresponding to a first subset of the plurality of different assays,
d) a labeling module to inform the system on the training vectors using parameters of the machine learning model to obtain output labels for the plurality of training samples,
e) a comparator module to compare the output labels to the known labels of the training samples,
f) a training module to iteratively search for optimal values of the parameters as part of training the machine learning model based on the comparing the output labels to the known labels of the training samples, and
g) an output module to provide the parameters of the machine learning model and the set of features for the machine learning model.

### VI. Methods of Classifying Subjects in a Population

The disclosed methods are directed to ascertaining parameters of genomic DNA expression associated with colon cell proliferative disorders via analysis of expressed miRNA in a subject. The method is for use in the improved diagnosis, treatment and monitoring of colon cell proliferative disorders, more specifically by enabling the improved identification of and differentiation between stages or subclasses of said disorder and the genetic predisposition to said disorders.

In some embodiments, the method comprises analyzing differential expression of miRNA in a biological sample from a subject in a population.

The present disclosure provides a method for detecting a colon cell proliferative disorder that may be applied to cell-free samples, e.g., to detect differentially-expressed cell-free miRNA between subjects with and without a colon cell proliferative disorder. The method utilizes detection of miRNA as the basic “positive” or “negative” for a colon cell proliferative disorder signal compared to a healthy subject not having a colon cell proliferative disorder.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In an aspect, the present disclosure provides a method for determining a miRNA profile of a biological sample from a subject comprising:
a) isolating RNA from the biological sample;
b) ligating RNA adapters to the nucleic acid from the biological sample before or after reverse transcribing the RNA to cDNA;
c) amplifying the cDNA of step b);
d) determining the nucleic acid sequence of the cDNA molecules, and
e) aligning the nucleic acid sequence of the nucleic acid molecules to a reference nucleic acid sequence for a pre-identified panel of miRNAs selected from the group listed in Tables 1-11, to determine the miRNA profile of the subject.

In some embodiments, a nucleic acid sequencing library is prepared before the amplification.

In some embodiments, the adapter ligation comprises RNA adapter ligation, adapter blocking, adapter circularization and dimer removal before c).

In some embodiments, the reference nucleic acid sequence is part of a human genome or human transcriptome database.

In some embodiments, determining the miRNA profile of the subject comprises generating a counts table of expressed miRNA.

In some embodiments, determining the miRNA profile of the subject comprises generating a counts table of expressed miRNA to identify differentially-abundant miRNA.

In some embodiments, the miRNA profile is associated with a colon cell proliferative disorder and provides classification of a subject as having a colon cell proliferative disorder.

In some embodiments, the biological sample obtained from the subject is selected from the group consisting of bodily fluids, stool, colonic effluent, urine, blood plasma, blood serum, whole blood, isolated blood cells, cells isolated from the blood, and combinations thereof.

In some embodiments, the method comprises applying the measured miRNA signature panel from the subject against a database of measured miRNA signature panels from healthy subjects, wherein the database is stored on a computer system; determining that the subject has an increased risk of having a colon cell proliferative disorder by measuring a change of at least 5%, at least 10%, at least 15%, at least 20%, at least 25%, at least 30%, at least 35%, at least 40%, at least 45%, or at least 50% in the miRNA signature panel relative to miRNA status from healthy subjects.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the colon cell proliferative disorder is selected from the group consisting of stage 1 colorectal cancer, stage 2 colorectal cancer, stage 3 colorectal cancer, and stage 4 colorectal cancer.

In another aspect, the present disclosure provides a method for detecting a colon cell proliferative disorder in a subject, comprising:
a) isolating RNA from the biological sample;
b) ligating RNA adapters to the RNA from the biological sample and reverse transcribing the RNA to cDNA;
c) amplifying the cDNA of step b);
d) determining the nucleic acid sequence of the cDNA molecules, and
e) aligning the nucleic acid sequence of a pre-identified panel of miRNAs selected from the group listed in Tables 1-11 to determine the miRNA profile of the subject, and
f) inputting the miRNA profile into a machine learning model trained to be capable of distinguishing between healthy subjects and subjects with a colon cell proliferative disorder to provide an output value associated with presence of a colon cell proliferative disorder, thereby indicating the presence of a colon cell proliferative disorder in the subject.

In some embodiments, the reference nucleic acid sequence is part of a human genome or human transcriptome database.

In some embodiments, determining the miRNA profile of the subject comprises generating a counts table of expressed miRNA.

In some embodiments, determining the miRNA profile of the subject comprises generating a counts table of expressed miRNA to identify differentially-abundant miRNA.

In some embodiments, the miRNA profile is inputted into a machine learning model to obtain a classifier capable of discriminating between two groups of subjects (e.g., healthy vs cancer, disease stage, advanced adenoma vs cancer).

In some embodiments, the biological sample obtained from the subject is selected from the group consisting of bodily fluids, stool, colonic effluent, urine, blood plasma, blood serum, whole blood, isolated blood cells, cells isolated from the blood, and combinations thereof.

In some embodiments, the method comprises applying the measured miRNA signature panel from the subject against a database of measured miRNA signature panels from healthy subjects, wherein the database is stored on a computer system; determining that the subject has an increased risk of having a colon cell proliferative disorder by measuring a change of at least 5%, at least 10%, at least 15%, at least 20%, at least 25%, at least 30%, at least 35%, at least 40%, at least 45%, or at least 50% in the miRNA expression of the miRNA signature panel relative to miRNA status from healthy subjects.

In some embodiments, the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

In some embodiments, the colon cell proliferative disorder is selected from the group consisting of stage 1 colorectal cancer, stage 2 colorectal cancer, stage 3 colorectal cancer, and stage 4 colorectal cancer.

The trained machine learning methods, models, and discriminate classifiers described herein may be applied toward various medical applications including cancer detection, diagnosis and treatment responsiveness. As models may be trained with subject metadata and analyte-derived features, the applications may be tailored to stratify subjects in a population and guide treatment decisions accordingly.

### Diagnosis

Methods and systems provided herein may perform predictive analytics using artificial intelligence-based approaches to analyze acquired data from a subject (patient) to generate an output of diagnosis of the subject having cancer (e.g., colorectal cancer, CRC). For example, the application may apply a prediction algorithm to the acquired data to generate the diagnosis of the subject having the cancer. The prediction algorithm may comprise an artificial intelligence-based predictor, such as a machine learning-based predictor, configured to process the acquired data to generate the diagnosis of the subject having the cancer.

The machine learning predictor may be trained using datasets e.g., datasets generated by performing RNA assays using the signature panels described herein on biological samples of subjects from one or more sets of cohorts of patients having cancer as inputs and known diagnosis (e.g., staging and/or tumor fraction) outcomes of the subjects as outputs to the machine learning predictor.

Training datasets (e.g., datasets generated by performing methylation assays using the signature panels described herein on biological samples of subjects) may be generated from, for example, one or more sets of subjects having common characteristics (features) and outcomes (labels). Training datasets may comprise a set of features and labels corresponding to the features relating to diagnosis. Features may comprise characteristics such as, for example, certain ranges or categories of cfRNA assay measurements, such as counts of cfRNA or fragments thereof in a biological sample obtained from a healthy and disease samples that overlap or fall within each of a set of bins (genomic windows) of a reference genome. For example, a set of features collected from a given subject at a given time point may collectively serve as a diagnostic signature, which may be indicative of an identified cancer of the subject at the given time point. Characteristics may also include labels indicating the subject's diagnostic outcome, such as for one or more cancers.

Labels may comprise outcomes such as, for example, a known diagnosis (e.g., staging and/or tumor fraction) outcomes of the subject. Outcomes may include a characteristic associated with the cancers in the subject. For example, characteristics may be indicative of the subject having one or more cancers.

Training sets (e.g., training datasets) may be selected by random sampling of a set of data corresponding to one or more sets of subjects (e.g., retrospective and/or prospective cohorts of patients having or not having one or more cancers). Alternatively, training sets (e.g., training datasets) may be selected by proportionate sampling of a set of data corresponding to one or more sets of subjects (e.g., retrospective and/or prospective cohorts of patients having or not having one or more cancers). Training sets may be balanced across sets of data corresponding to one or more sets of subjects (e.g., patients from different clinical sites or trials). The machine learning predictor may be trained until certain pre-determined conditions for accuracy or performance are satisfied, such as having minimum desired values corresponding to diagnostic accuracy measures. For example, the diagnostic accuracy measure may correspond to prediction of a diagnosis, staging, or tumor fraction of one or more cancers in the subject.

Examples of diagnostic accuracy measures may include sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting the cancer (e.g., colorectal cancer).

In an aspect, the disclosure provides a method of using a classifier capable of distinguishing a population of subjects comprising:
a) assaying RNA in the biological sample, wherein the assaying provides a set of measured values representative of the RNA in the biological sample,
b) identifying a set of features corresponding to properties of the RNA in the biological sample to be input to a machine learning or statistical model,
c) preparing a feature vector of feature values from each of the plurality of sets of measured values, each feature value corresponding to a feature of the set of features and including one or more measured values, wherein the feature vector comprises at least one feature value obtained using each set of the plurality of sets of measured values,
d) loading, into a memory of a computer system, the machine learning model comprising the classifier, the machine learning model trained using training vectors obtained from training biological samples, a first subset of the training biological samples identified as having a specified property and a second subset of the training biological samples identified as not having the specified property,
e) inputting the feature vector into the machine learning model to obtain an output classification of whether the biological sample has the specified property, thereby distinguishing a population of subjects having the specified property.

In another aspect, the present disclosure provides a method for identifying a cancer in a subject, comprising:
a) isolating RNA from the biological sample;
b) ligating RNA adapters to the RNA from the biological sample and reverse transcribing the RNA to cDNA;
c) amplifying the cDNA of step b);
d) determining the nucleic acid sequence of the cDNA molecules, and
e) aligning the nucleic acid sequence of the nucleic acid molecules to a reference nucleic acid sequence for a pre-identified panel of miRNAs selected from the group listed in Tables 1-11, to determine the miRNA profile of the subject,
f) inputting the miRNA profile into a machine learning model trained to be capable of distinguishing between healthy subjects and subjects with a colon cell proliferative disorder to provide an output value associated with presence of a colon cell proliferative disorder, thereby indicating the presence of a colon cell proliferative disorder in the subject to generate a likelihood of said subject having said cancer.

In some embodiments, said at least about 10 distinct miRNAs comprises at least about 20 distinct miRNAs, each of said at least about 20 distinct miRNAs comprising at least a portion of a miRNA listed in Tables 1-11. In some examples, said at least about 10 distinct miRNAs comprises at least about 30 distinct miRNAs, each of said at least about 30 distinct miRNAs comprising at least a portion of a miRNA listed in Tables 1-11.

Some embodiments provide a profile of differentially expressed miRNAs in a sample of a subject having colon cell proliferative disorder, or having high risk of developing colon cell proliferative disorder, particularly, when the subject has a pancreatic lesion. The profile of differentially expressed miRNAs in a sample of a subject having colon cell proliferative disorder, or having a high risk of developing colon cell proliferative disorder, comprises use of a profile of up-regulated/over-expressed miRNAs and a profile of down-regulated or under-expressed miRNAs.

In some embodiments, the method for detecting in a subject the presence of colon cell proliferative disorder, or a high risk of developing colon cell proliferative disorder, comprises:
a) detecting the level of expression of one or more miRNAs in a sample from the subject; and
b) comparing the detected expression level to a reference expression level, wherein a differential expression of the one or more miRNAs in the sample, as compared to the reference expression level, is indicative of the presence of colon cell proliferative disorder, or a higher risk of developing colon cell proliferative disorder, versus the absence of colon cell proliferative disorder, or a lower risk of developing colon cell proliferative disorder, respectively.

The differential expression of the one or more miRNAs in the sample, as compared to the reference expression level, may be indicative of a colon cell proliferative disorder precursor.

In some embodiments, the sample is a tissue sample, and the one or more miRNAs belong to a profile of miRNAs that are differentially expressed in a cell of a subject having a higher risk of developing colon cell proliferative disorder as compared to the corresponding cell of a subject having lower risk of developing colon cell proliferative disorder.

In some embodiments, the subject has a colorectal lesion and the one or more miRNAs belong to a profile of differentially expressed miRNAs in a sample of a subject having a colorectal lesion and having higher risk of developing colon cell proliferative disorder compared to the corresponding sample of a subject having a pancreatic lesion and having lower risk of developing colon cell proliferative disorder.

Some methods may be used for detecting the expression level of one or more miRNAs in a sample. For example, measurement of miRNA can be carried out by barcode-based assay, miRNA microarray analysis (e.g., chip), digital polymerase chain reaction (PCR), real-time PCR, quantitative reverse transcription PCR (qRT-PCR), semi-quantitative PCR, Northern blot, or in situ hybridization. For example, the mature miRNA is measured, for example, using an in vitro assay.

A variety of statistical and mathematical methods for establishing the threshold or cutoff level of expression may be used. A threshold or cutoff expression level for a particular biomarker may be selected, for example, based on data from Receiver Operating Characteristic (ROC) plots, such as described in the Examples and Figures of the present disclosure. In some embodiments, these threshold or cutoff expression levels can be varied, for example, by moving along the ROC plot for a particular biomarker or combinations thereof, to obtain different values for sensitivity or specificity thereby affecting overall assay performance. For example, if the objective is to have a robust diagnostic method from a clinical point of view, high sensitivity should be prioritized. However, if the goal is to have a cost-effective method, high specificity should be prioritized. The best cutoff refers to the value obtained from the ROC plot for a particular biomarker that produces the best sensitivity and specificity. Sensitivity and specificity values are calculated over the range of thresholds (cutoffs). Thus, the threshold or cutoff values can be selected such that the sensitivity and/or specificity are at least about 70%, and can be, for example, at least 75%, at least 80%, at least 85%, at least 90%, at least 95%, at least 96%, at least 97%, at least 98%, at least 99% or at least 100% in at least 60% of the patient population assayed, or in at least 65%, 70%, 75% or 80% of the patient population assayed.

Consequently, some of the embodiments of the present disclosure may be carried out by determining the expression levels of at least the microRNAs previously cited in a minimally-invasive sample isolated from the subject to be diagnosed or screened, and comparing the expression levels of said microRNAs with pre-determined threshold or cutoff values, wherein said pre-determined threshold or cutoff values correspond to the expression level of said microRNAs which correlates with the highest specificity at a desired sensitivity in a ROC curve calculated based on the expression levels of the microRNAs determined in a patient population being at risk of suffering colorectal cancer or colorectal adenoma, wherein the overexpression of at least one of said microRNAs with respect to said pre-determined cutoff value is indicative that the subject suffers from colorectal cancer or colorectal adenoma with said desired sensitivity.

As another example, such a pre-determined condition may be that the specificity of predicting the colon cell proliferative disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the colon cell proliferative disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the colon cell proliferative disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve of predicting the colon cell proliferative disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

### Treatment Responsiveness

The predictive classifiers, systems, and methods described herein may be applied toward classifying populations of individuals for a number of clinical applications (e.g., based on performing RNA assays using the signature panels described herein on biological samples of individuals). Examples of such clinical applications include, detecting early-stage cancer, diagnosing cancer, classifying cancer to a particular stage of disease, determining responsiveness or resistance to a therapeutic agent for treating cancer.

The methods and systems described herein may be applied to characteristics of a colon cell proliferative disorder, such as grade and stage. Therefore, combinations of analytes and assays may be used in the present systems and methods to predict responsiveness of cancer therapeutics across different cancer types in different tissues and classifying subjects based on treatment responsiveness. In some embodiments, the classifiers described herein are capable of stratifying a group of subjects into treatment responders and non-responders.

In another aspect, the present disclosure provides a method for monitoring minimal residual disease in a subject previously treated for disease comprising: determining a miRNA profile as described herein as a baseline miRNA state and repeating an analysis to determine the miRNA profile at one or more pre-determined time points wherein a change from baseline indicates a change in the minimal residual disease status at baseline in the subject.

In some embodiments, the minimal residual disease is selected from response to treatment, tumor load, residual tumor post-surgery, relapse, secondary screen, primary screen, and cancer progression.

In another aspect, a method is provided for determining response to treatment.

In another aspect, a method is provided for monitoring tumor load.

In another aspect, a method is provided for detecting residual tumor post-surgery.

In another aspect, a method is provided for detecting relapse.

In another aspect, a method is provided for use as a secondary screen.

In another aspect, a method is provided for use as a primary screen.

In another aspect, a method is provided for monitoring cancer progression.

The present disclosure also provides a method for determining a drug target of a condition or disease of interest (e.g., genes that are relevant or important for a particular class), comprising assessing a sample obtained from a subject for the level of gene expression for at least one gene; and using a neighborhood analysis routine, determining genes that are relevant for classification of the sample, to thereby ascertain one or more drug targets relevant to the classification.

The present disclosure also provides a method for determining the efficacy of a drug designed to treat a disease class, comprising obtaining a sample from an individual having the disease class; subjecting the sample to the drug; assessing the drug-exposed sample for the level of gene expression for at least one gene; and, using a computer model built with a weighted voting scheme, classifying the drug-exposed sample into a class of the disease as a function of relative gene expression level of the sample with respect to that of the model.

The present disclosure also provides a method for determining the efficacy of a drug designed to treat a disease class, wherein an individual has been subjected to the drug, comprising obtaining a sample from the individual subjected to the drug; assessing the sample for the level of gene expression for at least one gene; and using a model built with a weighted voting scheme, classifying the sample into a class of the disease including evaluating the gene expression level of the sample as compared to gene expression level of the model.

The present disclosure also provides a method of determining whether a subject belongs to a phenotypic class (e.g., intelligence, response to a treatment, length of life, likelihood of viral infection or obesity), comprising obtaining a sample from the subject; assessing the sample for the level of gene expression for at least one gene; and using a model built with a weighted voting scheme, classifying the sample into a class of the disease including evaluating the gene expression level of the sample as compared to gene expression level of the model.

In an aspect, the systems and methods described herein that relate to classifying a population based on treatment responsiveness refer to cancers that are treated with chemotherapeutic agents of the classes DNA damaging agents, DNA repair target therapies, inhibitors of DNA damage signaling, inhibitors of DNA damage induced cell cycle arrest and inhibition of processes indirectly leading to DNA damage, but not limited to these classes. Each of these chemotherapeutic agents may be considered a “DNA-damage therapeutic agent” as the term is used herein.

Based on a patient's analyte data, the patient may be classified into high-risk and low-risk patient groups, such as a patient with a high or low risk of clinical relapse, and the results may be used to determine a course of treatment. For example, a patient determined to be a high-risk patient may be treated with adjuvant chemotherapy after surgery. For a patient deemed to be a low-risk patient, adjuvant chemotherapy may be withheld after surgery. Accordingly, the present disclosure provides, in some aspects, a method for preparing a gene expression profile of a colon cancer tumor that is indicative of risk of recurrence.

In some examples, the classifiers described herein are capable of stratifying a population of subjects between responders and non-responders to treatment.

In another aspect, methods disclosed herein may be applied to clinical applications involving the detection or monitoring of cancer.

In some embodiments, methods disclosed herein may be applied to determine and/or predict response to treatment.

### Monitoring Colorectal Cancer

After using a trained algorithm to process the dataset, the colorectal cancer may be identified or monitored in the subject. The identification may be based at least in part on quantitative measures of sequence reads of the dataset at a panel of colorectal cancer-associated miRNA (e.g., quantitative measures of RNA transcripts). For example, the monitoring may comprise assessing the colorectal cancer of the subject at each of two or more different time points.

In some embodiments, methods disclosed herein may be applied to monitor and/or predict tumor load.

In some embodiments, methods disclosed herein may be applied to detect and/or predict residual tumor post-surgery.

In some embodiments, methods disclosed herein may be applied to detect and/or predict minimal residual disease post-treatment.

In some embodiments, methods disclosed herein may be applied to detect and/or predict relapse.

In an aspect, methods disclosed herein may be applied as a secondary screen.

In an aspect, methods disclosed herein may be applied as a primary screen.

In an aspect, methods disclosed herein may be applied to monitor cancer development.

In an aspect, methods disclosed herein may be applied to monitor and/or predict cancer risk.

The colorectal cancer may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the colorectal cancer by the trained algorithm may be calculated as the percentage of independent test samples (e.g., subjects known to have the colorectal cancer or subjects with negative clinical test results for the colorectal cancer) that are correctly identified or classified as having or not having the colorectal cancer.

The colorectal cancer may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the colorectal cancer using the trained algorithm may be calculated as the percentage of cell-free biological samples identified or classified as having the colorectal cancer that correspond to subjects that truly have the colorectal cancer.

The colorectal cancer may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the colorectal cancer using the trained algorithm may be calculated as the percentage of cell-free biological samples identified or classified as not having the colorectal cancer that correspond to subjects that truly do not have the colorectal cancer.

The colorectal cancer may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the colorectal cancer using the trained algorithm may be calculated as the percentage of independent test samples associated with presence of the colorectal cancer (e.g., subjects known to have the colorectal cancer) that are correctly identified or classified as having the colorectal cancer.

The colorectal cancer may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the colorectal cancer using the trained algorithm may be calculated as the percentage of independent test samples associated with absence of the colorectal cancer (e.g., subjects with negative clinical test results for the colorectal cancer) that are correctly identified or classified as not having the colorectal cancer.

In some embodiments, the trained algorithm may determine that the subject is at risk of colorectal cancer of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of colorectal cancer at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the colorectal cancer, the subject may be optionally provided with a therapeutic intervention (e.g., prescribing an appropriate course of treatment to treat the colorectal cancer of the subject). The therapeutic intervention may comprise a prescription of an effective dose of a drug, a further testing or evaluation of the colorectal cancer, a further monitoring of the colorectal cancer, or a combination thereof If the subject is currently being treated for the colorectal cancer with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the colorectal cancer. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a cell-free biological cytology, a FIT test, an FOBT test, or any combination thereof.

The quantitative measures of sequence reads of the dataset at the panel of colorectal cancer-associated genomic loci (e.g., quantitative measures of RNA transcripts at the colorectal cancer-associated genomic loci) may be assessed over a duration of time to monitor a patient (e.g., subject who has colorectal cancer or who is being treated for colorectal cancer). In such cases, the quantitative measures of the dataset of the patient may change during the course of treatment. For example, the quantitative measures of the dataset of a patient with decreasing risk of the colorectal cancer due to an effective treatment may shift toward the profile or distribution of a healthy subject (e.g., a subject without colorectal cancer). Conversely, for example, the quantitative measures of the dataset of a patient with increasing risk of the colorectal cancer due to an ineffective treatment may shift toward the profile or distribution of a subject with higher risk of the colorectal cancer or a more advanced colorectal cancer.

The colorectal cancer of the subject may be monitored by monitoring a course of treatment for treating the colorectal cancer of the subject. The monitoring may comprise assessing the colorectal cancer of the subject at two or more time points. The assessing may be based at least on the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined at each of the two or more time points.

In some embodiments, a difference in the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined between the two or more time points may be indicative of one or more clinical indications, such as (i) a diagnosis of the colorectal cancer of the subject, (ii) a prognosis of the colorectal cancer of the subject, (iii) an increased risk of the colorectal cancer of the subject, (iv) a decreased risk of the colorectal cancer of the subject, (v) an efficacy of the course of treatment for treating the colorectal cancer of the subject, and (vi) a non-efficacy of the course of treatment for treating the colorectal cancer of the subject.

In some embodiments, a difference in the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined between the two or more time points may be indicative of a diagnosis of the colorectal cancer of the subject. For example, if the colorectal cancer was not detected in the subject at an earlier time point but was detected in the subject at a later time point, then the difference is indicative of a diagnosis of the colorectal cancer of the subject. A clinical action or decision may be made based on this indication of diagnosis of the colorectal cancer of the subject, such as, for example, prescribing a new therapeutic intervention for the subject. The clinical action or decision may comprise recommending the subject for a secondary clinical test to confirm the diagnosis of the colorectal cancer. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a cell-free biological cytology, a FIT test, an FOBT test, or any combination thereof.

In some embodiments, a difference in the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined between the two or more time points may be indicative of a prognosis of the colorectal cancer of the subject.

In some embodiments, a difference in the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined between the two or more time points may be indicative of the subject having an increased risk of the colorectal cancer. For example, if the colorectal cancer was detected in the subject both at an earlier time point and at a later time point, and if the difference is a positive difference (e.g., the quantitative measures of sequence reads of the dataset at a panel of colorectal cancer-associated genomic loci (e.g., quantitative measures of RNA transcripts or DNA at the colorectal cancer-associated genomic loci) increased from the earlier time point to the later time point), then the difference may be indicative of the subject having an increased risk of the colorectal cancer. A clinical action or decision may be made based on this indication of the increased risk of the colorectal cancer, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the colorectal cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

In some embodiments, a difference in the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined between the two or more time points may be indicative of the subject having a decreased risk of the colorectal cancer. For example, if the colorectal cancer was detected in the subject both at an earlier time point and at a later time point, and if the difference is a negative difference (e.g., the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci decreased from the earlier time point to the later time point), then the difference may be indicative of the subject having a decreased risk of the colorectal cancer. A clinical action or decision may be made based on this indication of the decreased risk of the colorectal cancer (e.g., continuing or ending a current therapeutic intervention) for the subject. The clinical action or decision may comprise recommending the subject for a secondary clinical test to confirm the decreased risk of the colorectal cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

In some embodiments, a difference in the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined between the two or more time points may be indicative of an efficacy of the course of treatment for treating the colorectal cancer of the subject. For example, if the colorectal cancer was detected in the subject at an earlier time point but was not detected in the subject at a later time point, then the difference may be indicative of an efficacy of the course of treatment for treating the colorectal cancer of the subject. A clinical action or decision may be made based on this indication of the efficacy of the course of treatment for treating the colorectal cancer of the subject, e.g., continuing or ending a current therapeutic intervention for the subject. The clinical action or decision may comprise recommending the subject for a secondary clinical test to confirm the efficacy of the course of treatment for treating the colorectal cancer. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a cell-free biological cytology, a FIT test, an FOBT test, or any combination thereof

In some embodiments, a difference in the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci determined between the two or more time points may be indicative of a non-efficacy of the course of treatment for treating the colorectal cancer of the subject. For example, if the colorectal cancer was detected in the subject both at an earlier time point and at a later time point, and if the difference is a positive or zero difference (e.g., the quantitative measures of sequence reads of the dataset at a panel of colorectal cancer-associated genomic loci (e.g., quantitative measures of RNA transcripts or DNA at the colorectal cancer-associated genomic loci) comprising quantitative measures of a panel of colorectal cancer-associated genomic loci increased or remained at a constant level from the earlier time point to the later time point), and if an efficacious treatment was indicated at an earlier time point, then the difference may be indicative of a non-efficacy of the course of treatment for treating the colorectal cancer of the subject. A clinical action or decision may be made based on this indication of the non-efficacy of the course of treatment for treating the colorectal cancer of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the colorectal cancer. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a cell-free biological cytology, a FIT test, an FOBT test, or any combination thereof.

### VII. Kits

The present disclosure provides kits for identifying or monitoring a cancer of a subject. A kit may comprise probes or primers for identifying a quantitative measure (e.g., indicative of a presence, absence, or relative amount) of sequences at each of a plurality of cancer-associated genomic loci in a cell-free biological sample of the subject. A quantitative measure (e.g., indicative of a presence, absence, or relative amount) of sequences at each of a plurality of cancer-associated genomic loci in the cell-free biological sample may be indicative of one or more cancers. The probes may be selective for the sequences at the plurality of cancer-associated genomic loci in the cell-free biological sample. A kit may comprise instructions for using the probes to process the cell-free biological sample to generate datasets indicative of a quantitative measure (e.g., indicative of a presence, absence, or relative amount) of sequences at each of the plurality of cancer-associated genomic loci in a cell-free biological sample of the subject.

The probes in the kit may be selective for the sequences at the plurality of cancer-associated genomic loci in the cell-free biological sample. The probes in the kit may be configured to selectively enrich nucleic acid (e.g., RNA or DNA) molecules corresponding to the plurality of cancer-associated genomic loci. The probes in the kit may be nucleic acid primers. The probes in the kit may have partial or full sequence complementarity with nucleic acid sequences from one or more of the plurality of cancer-associated miRNA or fragments thereof. The plurality of cancer-associated miRNAs may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct cancer-associated miRNAs. The plurality of cancer-associated miRNA may comprise one or more members selected from the group consisting of miRNAs listed in Tables 1-11.

The instructions in the kit may comprise instructions to assay the cell-free biological sample using the probes that are selective for the sequences at the plurality of cancer-associated genomic loci in the cell-free biological sample. These probes may be nucleic acid molecules (e.g., RNA or DNA) having sequence complementarity with nucleic acid sequences (e.g., RNA or DNA) from one or more of the plurality of cancer-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the cell-free biological sample to generate datasets indicative of a quantitative measure (e.g., indicative of a presence, absence, or relative amount) of each of the plurality of cancer-associated miRNAs in the cell-free biological sample. A quantitative measure (e.g., indicative of a presence, absence, or relative amount) of sequences at each of a plurality of cancer-associated miRNAs in the cell-free biological sample may be indicative of one or more cancers.

The instructions in the kit may comprise instructions to measure and interpret assay readouts, which may be quantified at one or more of the plurality of cancer-associated genomic loci to generate the datasets indicative of a quantitative measure (e.g., indicative of a presence, absence, or relative amount) of sequences at each of the plurality of cancer-associated genomic loci in the cell-free biological sample. For example, quantification of array hybridization or polymerase chain reaction (PCR) corresponding to the plurality of cancer-associated miRNAs may generate the datasets indicative of a quantitative measure (e.g., indicative of a presence, absence, or relative amount) of miRNAs in the cell-free biological sample and differential expression between samples having known biological characteristics. Assay readouts may comprise quantitative PCR (qPCR) values, digital PCR (dPCR) values, digital droplet PCR (ddPCR) values, fluorescence values, etc., or normalized values thereof.

### EXAMPLES

### Example 1: miRNA Analysis in Cell Free Nucleic Acids

A total of 276 subjects were prospectively included in this study: 145 patients newly diagnosed with sporadic colorectal neoplasia (39 with CRC, 49 with Advanced Adenoma (AA), and 57 with Non-Advanced Adenomas) and 131 healthy individuals without personal history of any cancer and with a recent colonoscopy confirming the lack of colorectal neoplastic lesions. Patients with AA were those with adenomas having a size of at least 10 mm or histologically having high grade dysplasia or >20% villous component. Blood samples were collected prior to endoscopy or surgery in all individuals.

A description of the study cohort is provided in TABLE 12, which shows the number of healthy and cancer samples used for CRC experiments in the classification model (by stage, gender, and age).

TABLE 12

| CRC | | Cancer (n = 39) | AA (n = 49) | NAA (n = 57) | Contro1 (n = 131) |
|---|---|---|---|---|---|
| Gender | Female | 17 | 17 | 20 | 65 |
| | 119 (43.1%) | | | | |
| | Male | 22 | 32 | 37 | 66 |
| | 157 (56.9%) | | | | |
| Age | Median, | Median age: 61 | Median age: 63 | Median age: 63 | Median age: 59.0 |
| | IQR | IQR: 55.0-68.5 | IQR: 53.0-67.0 | IQR: 56.0-68.0 | IQR: 54.0-67.5 |

For each of 276 individuals, RNA was extracted from 100 microliters (uL) of plasma using MagMax mirVana Total RNA Isolation Kits. During extraction, known quantities of 52 synthetic miRNAs were added as spike ins to each plasma sample.

### Construction of Sequencing Libraries

### A. Library Construction

Cell-free nucleic acid samples were subjected to library preparation for next generation sequencing. For next generation sequencing approaches used herein, “library preparation” includes end-repair, A-tailing, adapter ligation, or any other preparation performed on the cell free RNA to permit subsequent sequencing of RNA. In some examples, a prepared cell-free nucleic acid library sequence contains adapters, sequence tags, index barcodes that are ligated onto cell-free nucleic acid sample molecules. Various commercially available kits are available to facilitate library preparation for next-generation sequencing approaches. Next generation sequencing library construction involves preparing nucleic acids targets using a coordinated series of enzymatic reactions to produce a random collection of RNA fragments, of specific size, for high throughput sequencing. Advances and the development of new library preparation technologies have expanded the application of next-generation sequencing to fields such as transcriptomics and epigenetics.

In some examples, library preparation kits may be selected from Nextera Flex (Illumina), IonAmpliseq (Thermo Fisher Scientific), and Genexus (Thermo Fisher Scientific), Agilent ClearSeq (Agilent), Agilent SureSelect Capture (Agilent), RealSeq (Realseq Biosciences), Archer FusionPlex (Illumina), BiooScientific NEXTflex (Perkin Elmer), IDT xGen (Illumina), Illumina TruSight (Illumina), SMARTer smRNA-Seq (Takara), Nimblegene SeqCap (Illumina), Qiaseq (Qiagen), or Qiagen GeneRead (Qiagen).

RealSeq® (RealSeq Biosciences; formerly Somagenics) is a method for preparing small-RNA sequencing libraries that greatly reduces incorporation bias in Next Generation Sequencing (NSG). This technology solves the problem of commonly used sequencing library preparations that lead to underdetection of many miRNAs, some by as much as 10,000-fold. Most bias stems from sequence-dependent variability in the enzymatic ligation reactions that attach the two adapters to the 3′ and 5′ ends of the miRNAs/small RNAs during preparation of sequencing libraries. By using a novel single adapter and circularization, RealSeq® greatly reduces library preparation bias.

### B. Sequencing

The sequencing libraries were diluted to 2 nanomolar (nM). Samples were loaded onto an Illumina NextSeqinstrument for sequencing to a depth of approximately 10 million reads per individual.

### C. RealSeq Analysis Summary

Sequence reads were trimmed to remove the RealSeq specific adapter and other primers and adapters using trimming software such as Trimmomatic, Skewer, SeqPurge, and Atropos. Trimmed reads were then aligned using the bowtie2 alignment software to a custom transcriptome made up of mature miRNA sequences, tRNA, rRNA, mitochondrial RNA, U snRNAs, Y RNAs, and synthetic spike-in sequences. samtools idxstats was used to count the number of reads mapping to each feature.

Various data preprocessing steps were performed to make the data more featurized for analysis. Data preprocessing has the objective to add missing values, aggregate information, label data with categories (data binning) and to smooth a trajectory. More advanced techniques like principle component analysis and feature selection were performed with statistical formulas and were applied to complex datasets.

Samples with less than 300,000 reads mapping to hairpin sequences were removed from subsequent analyses. Features with counts <25 across all samples were also removed. Several normalization strategies were compared including: mean of ratios normalization, trimmed mean of M-values, RUVseq, and log2CPM. Mean of ratios normalization method was used to normalize the data.

The sequences were trimmed using custom scripts to remove the Illumina adaptors from the miRNA sequences. The miRNA sequences were then mapped using bowtie to (1) the human genome, (2) a custom transcriptome including all known mature human miRNAs, and (3) a second transcriptome of hairpin miRNA sequences, as well as to a list of 52 synthetic, non-human derived, miRNAs. From the mapping locations, a counts table of both miRNA and miRNA hairpins were generated from each individual.

Using these count datasets, miRNAs that are differentially abundant between the plasma of healthy individuals vs. individuals with colorectal cancer or advanced adenomas were identified. Both count matrices were normalized using multiple methods including: (1) trimmed mean of M-values, (2) mean of ratios, and (3) RUVseq. Each of these three methods was run on the total RNA library to generate normalization factors as well as on the synthetic RNA spike ins to generate a second set of normalization factors. Normalized counts from each method were separately used to generate models identifying sequences displaying significantly different mature miRNAs and miRNA hairpins abundances between health states. Hairpins and miRNAs with a p<0.05 in 2 or more methods were considered strongly supported and were identified as markers for CRC and/or AA.

For classification models, the same normalizations were used on both datasets (mature miRNAs and miRNA hairpins) with the addition of Combat as a fourth normalization method. Normalized counts were also adjusted for known confounders including plasma quality, age, sex, and collection site. From the resultant counts dataset, a k-best model was constructed with k=8. The model was run on 500 independent seeds each splitting the data into 4 folds, training on 3 folds of the data and testing on the final, 4th, fold. The 8 features identified in each model were then compared to the list of differentially abundant miRNAs and hairpins.

### Example 2: Generating a Classification Signature for Colorectal Cancer and Advanced Adenoma

The evaluated machine learning methods comprised a series of transformations, in some cases including dimensionality reduction, followed by a supervised classification algorithm.

The purpose of a cross-validation (CV) procedure in assessing a classification model is to estimate a model's performance on new, previously unseen data that were not used to construct the model. The goal is to provide an approximation by repeatedly training a model on a distinct subset of the data and testing on a held-out subset of data, unseen by the model during training. K-fold cross-validation procedure requires dividing the entire dataset into k groups. For each of the k groups (or folds), a machine learning model was trained with the other k-1 folds, and the held-out fold is used as the test set. Stratified k-fold cross-validation stratifies the samples by class before dividing into folds so that the approximate proportion of samples is roughly equivalent across folds.

A number of models were trained with k-fold cross-validation (k=5); subsequently, the best performing model was evaluated with additional cross-validation procedures. The top 10 features were selected based on the ANOVA F-statistic during cross validation. Logistic Regression was used to classify samples. 100 different random seeds were used to split data into folds, therefore a total of 500 models were run (100 seeds, 5 folds per seed)

miRNAs were selected during feature selection. The column number of seeds shows the number of times a miRNA was picked during feature selection in the 500 models that were run. A high number, for example 499, for hsa-mir-889 indicates that this miRNA was selected in 499 out of the 500 models.

First, outliers (defined as feature values of a given sample that were above the 99th percentile of that feature across all training samples) were imputed to the 99th percentile value. Each feature was subsequently standardized across all training samples by subtracting the mean and dividing by the standard deviation. The same outlier replacement, using the means and standard deviations of the training set, were used to standardize the test set. If a dimensionality reduction transformation method was selected, it was trained on the training set and applied to all samples in both the training and the test sets. The dimensionality reduction transformations used in this study were truncated singular value decomposition (SVD) and principal component analysis (PCA).

Two possible classification algorithms were trained on the transformed input: logistic regression and support vector machine (SVM). Multiple hyperparameters were considered for each method using a random search of 100 iterations per fold with a validation set comprising a randomly selected 20% of the training data; the hyperparameters corresponding to the best performing validation set were selected to train a machine learning model to evaluate the test fold. Logistic regression had two hyperparameters: the inverse of regularization strength, and the choice of either L₁ or L₂ penalty. TABLE 13 shows a collection of miRNAs identified in cell-free nucleic acid samples as being associated with individuals having colorectal cancer in k-fold cross validation, along with associated average coefficient values for logistic regression (e.g., an average for the number of seeds that the miRNA was selected in).

TABLE 13

| | | Average coefficient value |
|---|---|---|
| | | for logistic regression |
| | | (average for the number |
| | Number of seeds selected | of seeds that the miRNA |
| miRNA | in (out of 500) | was selected in) |
| hsa-mir-889 | 499 | 0.01227171412 |
| hsa-mir-543 | 392 | 0.02242937901 |
| hsa-mir-376b | 366 | 0.001838778481 |
| hsa-mir-335 | 342 | 0.00000399410613 |
| hsa-mir-1185-1 | 262 | −0.008112394768 |
| hsa-mir-548k | 233 | 0.03467194037 |
| hsa-mir-12135 | 208 | 0.1467320116 |
| hsa-mir-369 | 168 | −0.001607343291 |
| hsa-mir-190a | 165 | 0.001031765066 |
| hsa-mir-6770-1 | 143 | 0.09696291942 |
| hsa-mir-382 | 135 | 0.003105079756 |
| hsa-mir-1843 | 129 | 0.001999311556 |
| hsa-mir-142 | 112 | −0.00002348964374 |
| hsa-mir-485 | 110 | 0.005757528395 |
| hsa-mir-548ax | 98 | 0.01063443146 |
| hsa-mir-548e | 98 | 0.06965708027 |
| hsa-mir-548al | 89 | 0.05388619835 |
| hsa-mir-548am | 89 | 0.001803661864 |
| hsa-mir-590 | 82 | −0.0005913171732 |
| hsa-mir-135a-2 | 81 | 0.209409359 |
| hsa-mir-6770-3 | 79 | 0.130261975 |
| hsa-mir-410 | 77 | −0.000443395015 |
| hsa-mir-376a-1 | 72 | 0.00006790511459 |
| hsa-mir-377 | 69 | −0.003048876316 |
| hsa-mir-570 | 62 | 0.01211643368 |
| hsa-mir-381 | 55 | 0.007964852332 |
| hsa-mir-665 | 48 | 0.01925932227 |
| hsa-mir-758 | 46 | 0.06822055355 |
| hsa-mir-6511a-3 | 43 | −0.03469441469 |
| hsa-mir-376a-2 | 42 | −0.0004471845472 |
| hsa-mir-155 | 38 | 0.004215096372 |
| hsa-mir-3140 | 38 | 0.07299821889 |
| hsa-mir-1277 | 34 | 0.006571441215 |
| hsa-mir-340 | 32 | −0.001637170648 |
| hsa-mir-548n | 28 | 0.03937433917 |
| hsa-mir-518b | 27 | 0.0866790012 |
| hsa-mir-654 | 26 | −0.003946375455 |
| hsa-mir-5581 | 23 | 0.1074504023 |
| hsa-mir-409 | 22 | −0.002519196915 |
| hsa-mir-628 | 21 | 0.003309890234 |
| hsa-mir-10399 | 19 | 0.01400376339 |
| hsa-mir-3184 | 19 | 0.0005741457151 |
| hsa-mir-423 | 14 | −0.0007777398231 |
| hsa-mir-548z | 12 | 0.002962701142 |
| hsa-mir-374a | 11 | 0.0009378886457 |
| hsa-mir-548a-3 | 11 | 0.01152914924 |
| hsa-mir-6770-2 | 10 | 0.006885855981 |
| hsa-mir-1185-2 | 9 | 0.003843378367 |
| hsa-mir-6077 | 9 | 0.1848254301 |
| hsa-mir-3202-1 | 8 | −0.1448356476 |
| hsa-mir-548o-2 | 8 | −0.01159264787 |
| hsa-mir-3143 | 7 | 0.02527664699 |
| hsa-mir-5009 | 7 | 0.1928650892 |
| hsa-mir-548g | 7 | 0.08575802863 |
| hsa-mir-656 | 7 | 0.01393539188 |
| hsa-mir-6818 | 7 | 0.08412729176 |
| hsa-mir-548l | 6 | 0.008159810018 |
| hsa-mir-548v | 6 | 0.06831132882 |
| hsa-mir-106b | 5 | 0.0001420887548 |
| hsa-mir-133b | 5 | 0.2074284074 |
| hsa-mir-548h-2 | 5 | 0.08619580177 |
| hsa-mir-136 | 4 | −0.003615557403 |
| hsa-mir-26b | 4 | 0.00001385575386 |
| hsa-mir-433 | 4 | 0.006869534333 |
| hsa-mir-4719 | 4 | 0.3432401024 |
| hsa-mir-3610 | 3 | 0 |
| hsa-mir-374b | 3 | −0.004197640226 |
| hsa-mir-376c | 3 | −0.0009238950727 |
| hsa-mir-4779 | 3 | 0.1750502307 |
| hsa-mir-496 | 3 | 0.005980723795 |
| hsa-mir-622 | 3 | 0.1956975342 |
| hsa-mir-671 | 3 | −0.007865086858 |
| hsa-mir-6876 | 3 | 0.1454046126 |
| hsa-let-7b | 2 | −0.000008662808689 |
| hsa-mir-103a-1 | 2 | −0.001822456296 |
| hsa-mir-103a-2 | 2 | 0.001520027525 |
| hsa-mir-10400 | 2 | 0.1984508462 |
| hsa-mir-135a-1 | 2 | 0.05744402254 |
| hsa-mir-139 | 2 | 0.0005742609812 |
| hsa-mir-191 | 2 | −0.00001105403881 |
| hsa-mir-2392 | 2 | 0.2984791466 |
| hsa-mir-26a-2 | 2 | −0.00002437884444 |
| hsa-mir-320c-2 | 2 | 0.001248926099 |
| hsa-mir-4468 | 2 | 0.06782728547 |
| hsa-mir-4484 | 2 | 0.01846424611 |
| hsa-mir-4521 | 2 | 0.1751726514 |
| hsa-mir-4706 | 2 | 0.1722001142 |
| hsa-mir-487a | 2 | 0.001771573345 |
| hsa-mir-548ac | 2 | 0.06019265588 |
| hsa-mir-5588 | 2 | 0.02659738315 |
| hsa-mir-579 | 2 | 0.001881716483 |
| hsa-mir-6734 | 2 | −0.0009272692234 |
| hsa-mir-6882 | 2 | 0.07055954999 |
| hsa-mir-9-1 | 2 | 0.01230175494 |
| hsa-mir-93 | 2 | 0.00001165527636 |
| hsa-let-7i | 1 | −0.00006929515082 |
| hsa-mir-103b-1 | 1 | 0.001366745737 |
| hsa-mir-103b-2 | 1 | −0.0004961485176 |
| hsa-mir-12136 | 1 | 0.0006137906358 |
| hsa-mir-1283-2 | 1 | 0.1251113007 |
| hsa-mir-1287 | 1 | 0.0217592237 |
| hsa-mir-130a | 1 | −0.00002556546956 |
| hsa-mir-146a | 1 | −0.0000105260071 |
| hsa-mir-146b | 1 | 0.0003532849175 |
| hsa-mir-151b | 1 | 0.0002453616989 |
| hsa-mir-154 | 1 | 0.001228707094 |
| hsa-mir-18a | 1 | −0.0005226497861 |
| hsa-mir-2278 | 1 | 0.006264706585 |
| hsa-mir-2355 | 1 | 0.009943231933 |
| hsa-mir-3138 | 1 | 0.102350404 |
| hsa-mir-3168 | 1 | −0.1457401201 |
| hsa-mir-3202-2 | 1 | −0.1529618687 |
| hsa-mir-320c-1 | 1 | −0.003100822914 |
| hsa-mir-320d-1 | 1 | 0.003877366341 |
| hsa-mir-320d-2 | 1 | 0.005518469682 |
| hsa-mir-324 | 1 | 0.001810403358 |
| hsa-mir-331 | 1 | −0.001317093481 |
| hsa-mir-3529 | 1 | 0.002602265853 |
| hsa-mir-374c | 1 | 0.008427415 |
| hsa-mir-431 | 1 | 0 |
| hsa-mir-450b | 1 | 0.02620627163 |
| hsa-mir-4651 | 1 | 0 |
| hsa-mir-4716 | 1 | −0.1316026223 |
| hsa-mir-4763 | 1 | −0.09201122096 |
| hsa-mir-4772 | 1 | 0.03507199649 |
| hsa-mir-548a-1 | 1 | 0.05655605303 |
| hsa-mir-548ah | 1 | 0.160519459 |
| hsa-mir-548aj-1 | 1 | 0.006007596705 |
| hsa-mir-5703 | 1 | 0.1132904105 |
| hsa-mir-574 | 1 | 0.003831642737 |
| hsa-mir-6729 | 1 | −0.2068859174 |
| hsa-mir-6781 | 1 | 0.1265329942 |
| hsa-mir-6802 | 1 | 0.1171690204 |
| hsa-mir-7-1 | 1 | 0 |
| hsa-mir-7111 | 1 | 0.0425464288 |
| hsa-mir-9902-1 | 1 | 0.01191197396 |

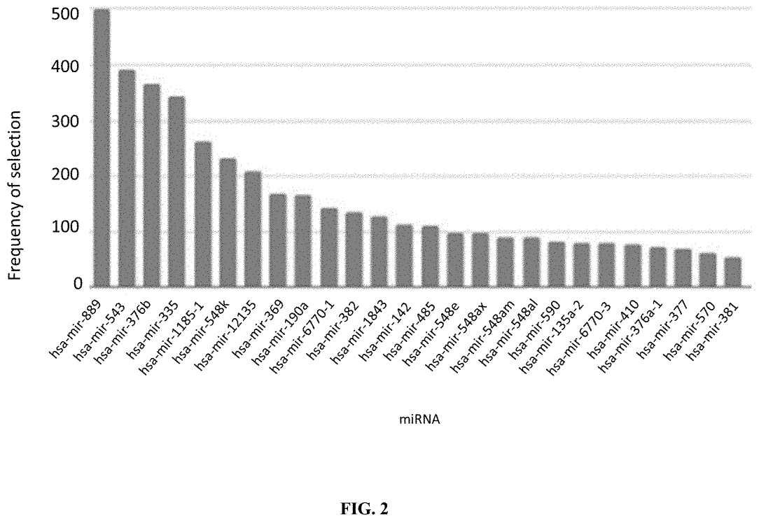

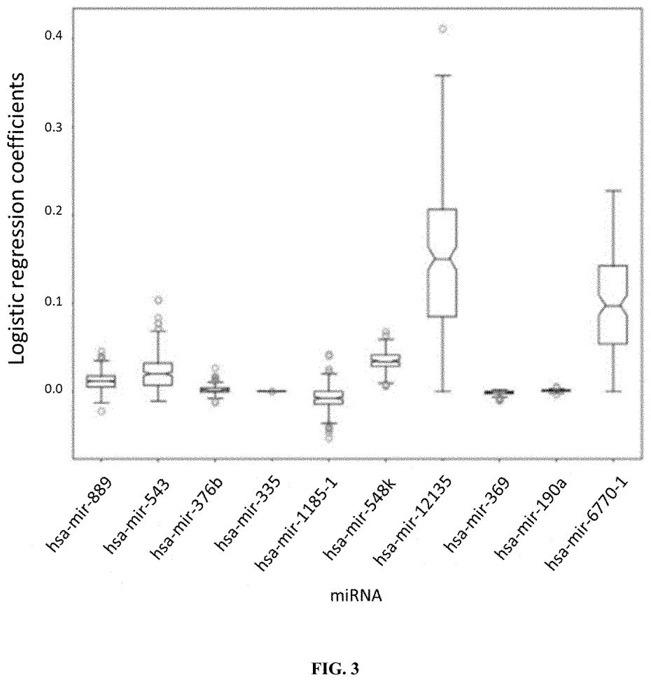

In total, 138 miRNAs were found to be associated with colorectal cancer. Not all identified miRNAs were necessary to be included in a classification model in order to distinguish between healthy individuals and individuals with colorectal cancer. Thus, some regions appear to be generally indicative of the various types of cancers assessed. Other miRNAs are more frequent in subgroups of colorectal cancer. In the context of this assay and the types of cancers examined, certain miRNAs may be described as being “specifically associated with colorectal cancer” with a higher frequency in the CV and carry a higher weight in the signature when the sample sequences were trained in a predictive model. These higher frequency miRNAs associated with colorectal cancer are used in specific models trained to discriminate populations of individuals between healthy and CRC. FIG. 2 provides a histogram showing miRNAs selected during a feature selection. The bars represent the number of models (max=500) in which the miRNA was selected. FIG. 3 provides a graph showing logistic regression coefficients of the top 10 most frequently selected miRNAs.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-57. (canceled)

58. A method for determining a micro ribonucleic acid (miRNA) profile of a biological sample from a subject, comprising:
a) isolating RNA molecules from the biological sample;
b) ligating RNA adapters to the RNA molecules or to complementary deoxyribonucleic acid (cDNA) molecules derived from the RNA molecules, wherein the RNA adapters are ligated before or after reverse transcribing the RNA molecules to the cDNA molecules;
c) amplifying the cDNA molecules;
d) determining nucleic acid sequences of the cDNA molecules;
e) aligning the nucleic acid sequences to a reference human genome or reference human transcriptome at a set of genomic loci corresponding to a panel of miRNAs selected from the group listed in Tables 1-11; and
f) determining the miRNA profile of the subject based at least in part on the aligned nucleic acid sequences.

59. The method of claim 58, wherein the panel of miRNAs comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 250 miRNAs selected from the group listed in Tables 1-11.

60. The method of claim 58, wherein the panel of miRNAs is characteristic of advanced adenoma, and wherein the panel comprises: a) hsa-miR-1273a, hsa-miR-17-5p, hsa-miR-20a-3p, and hsa-miR-20b-5p; b) hsa-miR-3065-5p, hsa-miR-4785, hsa-miR-5096, and hsa-miR-5189-5p, or c) hsa-miR-545-3p, hsa-miR-570-3p, hsa-miR-624-3p, hsa-mir-1181, and hsa-mir-6073, wherein the miRNAs are differentially expressed between a biological sample from a subject having advanced adenoma or subtype thereof, and a biological sample from a subject without advanced adenoma or subtype thereof.

61. The method of claim 58, wherein the panel of miRNAs is characteristic of colorectal cancer, and wherein the panel comprises: a) hsa-miR-1250-5p, hsa-miR-1255a, hsa-miR-223-3p, hsa-miR-338-3p, and hsa-miR-338-5p; b) hsa-miR-424-5p, hsa-miR-424-3p, hsa-miR-450a-5p, hsa-miR-450b-5p, and hsa-miR-4772-3p; c) hsa-miR-4772-5p, hsa-miR-625-5p, hsa-miR-7847-3p, hsa-miR-1181, hsa-miR-3651, and hsa-mir-6073; d) hsa-mir-6125, hsa-mir-7704, hsa-miR-19b-3p, hsa-miR-19a-3p, and hsa-miR-3157-5p; e) hsa-miR-142-3p, hsa-miR-30c-5p, hsa-miR-6741-5p, hsa-miR-590-3p, and hsa-miR-4685-5p; f) hsa-miR-3648, hsa-miR-331-3p, hsa-miR-1303, hsa-miR-6790-3p, hsa-miR-6867-5p, and hsa-miR-942-5p; g) hsa-miR-378a-3p, hsa-miR-1287-5p, hsa-mir-4785, hsa-miR-324-3p, and hsa-miR-550b-2-5p; h) hsa-miR-200c-3p, hsa-miR-200b-3p, hsa-miR-3679-5p, hsa-miR-550a-3-5p, and hsa-miR-3187-3p; i) hsa-miR-181b-5p, hsa-miR-3138, hsa-miR-146a-5p, hsa-miR-6721-5p, hsa-miR-23b-3p, and hsa-miR-28-5p; j) hsa-miR-320d, hsa-miR-940, hsa-miR-320d-1, hsa-miR-10a-5p, and hsa-miR-340-5p; k) hsa-miR-320b, hsa-miR-335-5p, hsa-miR-320c, hsa-miR-501-3p, and hsa-miR-548n; or l) hsa-miR-27a-3p, hsa-miR-3065-3p, hsa-miR-548aa@, hsa-miR-584-3p, and hsa-miR-22-3p, wherein the miRNAs are differentially expressed between a biological sample from a subject having the colorectal cancer or subtype thereof, and a biological sample from a subject without the colorectal cancer or subtype thereof.

62. The method of claim 58, further comprising enriching or depleting the RNA molecules or the cDNA molecules.

63. The method of claim 58, further comprising preparing a miRNA library before the amplifying.

64. The method of claim 58, wherein the RNA adapters are ligated before the reverse transcribing the RNA molecules to the cDNA molecules.

65. The method of claim 58, wherein the RNA adapters are ligated after the reverse transcribing the RNA molecules to the cDNA molecules.

66. The method of claim 58, wherein the ligating further comprises incorporating sample-specific barcodes and/or molecule-specific unique molecular identifiers (UMIs).

67. The method of claim 58, wherein the ligating further comprises performing adapter blocking, adapter circularization, and dimer removal.

68. The method of claim 58, wherein the ligating further comprises performing 3′ RNA adapter ligation, 5′ RNA adapter ligation, reverse transcription with unique molecular identifier (UMI) assignment, and cDNA cleanup.

69. The method of claim 58, further comprising performing one or more of: 1) extracting RNA molecules from the biological sample followed by direct RNA counting, 2) extracting RNA molecules from the biological sample followed by A tailing, then reverse transcribing to the cDNA with template switching, 3) extracting RNA molecules from the biological sample followed by A tailing, then performing reverse transcription polymerase chain reaction (RT-PCR) and quantitative PCR (qPCR) or digital droplet PCR (ddPCR), 4) extracting RNA molecules from the biological sample followed by sequence-specific ligation, and then RT-PCR and qPCR or ddPCR, and 5) extraction-free miRNA profiling of RNA molecules from the biological sample in absence of performing RNA isolation.

70. The method of claim 69, wherein determining the miRNA profile further comprises generating a counts table of expressed miRNA.

71. The method of claim 69, wherein determining the miRNA profile further comprises generating a counts table normalized based on expressed miRNA to identify differentially-abundant miRNA.

72. The method of claim 69, wherein the miRNA profile is associated with a colon cell proliferative disorder and provides classification of the subject as having the colon cell proliferative disorder or not having the colon cell proliferative disorder.

73. The method of claim 72, wherein the colon cell proliferative disorder is selected from the group consisting of adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

74. The method of claim 73, wherein the advanced adenoma comprises a tubular adenoma, a tubulovillous adenoma, a villous adenoma, an adenocarcinoma, or a hyperplastic polyp.

75. The method of claim 72, wherein the colon cell proliferative disorder is selected from the group consisting of stage 1 colorectal cancer, stage 2 colorectal cancer, stage 3 colorectal cancer, and stage 4 colorectal cancer.

76. The method of claim 58, wherein the biological sample from the subject is selected from the group consisting of bodily fluid, stool, colonic effluent, urine, blood plasma, blood serum, whole blood, isolated blood cells, cells isolated from the blood, and a combination thereof.

77. The method of claim 58, further comprising comparing the miRNA profile against a database of reference miRNA profiles from healthy subjects; and determining that the subject has an increased risk of having a colon cell proliferative disorder based at least in part on measuring a change of at least 15% in miRNA expression of the miRNA profile relative to the reference miRNA profiles.

78. The method of claim 58, further comprising computer processing the miRNA profile (i) using a machine learning classifier that is trained to distinguish between subjects having a colon cell proliferative disorder and subjects not having the colon cell proliferative disorder, and generating an output indicative of subjects having the colon cell proliferative disorder or subjects not having the colon cell proliferative disorder, or (ii) against a reference miRNA profile, thereby detecting a presence or an absence of the colon cell proliferative disorder in the subject.

79. The method of claim 78, further comprising computer processing the miRNA profile using the machine learning classifier.

80. The method of claim 78, further comprising computer processing the miRNA profile against the reference miRNA profile.

81. The method of claim 78, wherein the machine learning classifier comprises sets of measured values describe characteristics of the differential miRNA expression selected from the group consisting of: count or rate of observing fragments with different counts, raw miRNA abundance, miRNA abundance normalized to housekeeping genes, miRNA abundance normalized to synthetic sequences, log normalized miRNA abundance, fragment length, fragment midpoint, read mapping position and read pile up along mature miRNAs or miRNA hairpins, and abundances of clusters of miRNAs.

82. The method of claim 78, wherein the machine learning classifier is trained using training data obtained from training biological samples, wherein a first subset of the training biological samples is identified as corresponding to a subject having the colon cell proliferative disorder and a second subset of the training biological samples is identified corresponding to a subject as not having the colon cell proliferative disorder.

83. The method of claim 78, wherein the machine learning classifier is selected from the group consisting of a deep learning classifier, a neural network classifier, a linear discriminant analysis (LDA) classifier, a quadratic discriminant analysis (QDA) classifier, a support vector machine (SVM) classifier, a random forest (RF) classifier, a linear kernel support vector machine classifier, a first or second order polynomial kernel support vector machine classifier, a ridge regression classifier, an elastic net algorithm classifier, a sequential minimal optimization algorithm classifier, a naive Bayes algorithm classifier, a principal component analysis classifier, and a combination thereof.

84. The method of claim 78, further comprising administering a treatment to the subject based on the detected presence of the colon cell proliferative disorder in the subject.

85. The method of claim 58, further comprising monitoring minimal residual disease in the subject, wherein the subject has been previously treated for a disease, based at least in part by on a difference between the miRNA profile and a baseline miRNA profile from a previous time point.
